(19) United States
(12) Patent Application Publication
(10) Pub. No.: US 2026/0202423 A1
(43) Pub. Date: Jul. 16, 2026

(54) MARKERS FOR PROGNOSING AN INCREASED RISK OF EARLY ONSET PREECLAMPSIA

(71) Applicant: Oxford University Innovation Limited, Oxford (GB)

(72) Inventors: Wei ZHANG, Oxford (Oxfordshire) (GB); Shuhan JIANG, Oxford (Oxfordshire) (GB); Manu VATISH, Oxford (Oxfordshire) (GB); Masyuki ONO, Kanagawa (JP); Kenichi FUJIMAKI, Hamburg (DE); Atsushi SAITO, Kanagawa (JP); Makoto ITONAGA, Kanagawa (JP); Kenta ODA, Hamburg (DE)

(21) Appl. No.: 19/136,573

(22) PCT Filed: Dec. 15, 2023
(86) PCT No.: PCT/GB2023/053270

§ 371 Date: Jun. 6, 2025

(30) Foreign Application Priority Data
Dec. 16, 2022 (GB) 2219102.7

(51) Int. Cl. G01N 33/68; G01N 21/47; G01N 33/543; G01N 33/573

(52) U.S. Cl. CPC G01N 33/689; G01N 21/4738; G01N 33/54346; G01N 33/54386; G01N 33/573; G01N 2333/4731; G01N 2333/70596; G01N 2333/916; G01N 2800/368; G01N 2800/50; G01N 2800/52

(57) ABSTRACT

The invention provides methods for prognosing an increased risk of early onset preeclampsia (EOPE) in a pregnant subject before the disease onset. The methods comprises quantifying small extracellular vesicles that express pairs of biomarkers selected from CD10, CD63 and placental alkaline phosphatase (PLAP) in a sample obtained from the subject.

### FIELD

The invention relates to methods for prognosing an increased risk of early onset preeclampsia (EOPE) in a pregnant subject before the disease onset.

### BACKGROUND

Extracellular vesicles (EVs) are cell-derived particles bound by a lipid bilayer, and play important roles in intercellular communication by transferring different cargo including proteins, lipids and genetic material to recipient cells. EVs have been detected in various biofluids such as plasma, urine, bile, saliva, breast milk, semen and cerebrospinal fluid. So far, small EVs (sEVs) have been found to play important roles in physiological functions such as angiogenesis, antigen presentation, intercellular signalling. sEVs are also considered as biomarkers for a variety of diseases, such as cancers, neurodegenerative disease, kidney injuries, hepatitis.

Depending on their biogenesis, EVs can be classified into endosomal-origin (exosomes) and plasma membrane budding-origin (microvesicles), where the former is formed by inward budding of the endosomal membrane during maturation of multivesicular endosomes (MVEs), and the latter involves directly outward budding at the plasma membrane. As a result, both types of EVs share cell plasma membrane associated molecules and genetic makeups of their cell origin although exosomes are preferentially enriched for one or multiple tetraspanins (CD63, CD9 and CD81) in a cell type specific manner (1). Plasma membrane associated molecules have been instrumental not only in the identification of the cellular origin of EVs but also identification of disease-associated molecules, so-called biomarkers. Distinguishing EV phenotype on their biogenesis pathway however, has been challenging. For this reason, the ISEV has recommended an EV classification based on the size of the EVs, where EVs can be classified into medium/large EVs (>200 nm), small EVs (sEVs) (<200 nm).

The human placenta, an organ of fetal origin, anchors into the maternal uterus and is responsible for the exchange of nutrients, waste and gases throughout pregnancy. The maternal surface of the placenta, the syncytiotrophoblast (STB), is in direct contact with maternal blood and releases placental EVs into the maternal circulation (2). Placental EVs carry a unique marker of their origin, placental alkaline phosphatase (PLAP), which has been used to delineate these vesicles from those of other sources. Using PLAP, placental EVs have been reported as being detectable in maternal circulation as early as the first trimester in normal pregnancy (2). It has been shown throughout normal pregnancy, that the level of placental EVs in the maternal circulation increases with advancing gestational age (3). Placental EVs have been reported to regulate the functions of maternal endothelial cells, immune cells and hepatocytes (4-6). Thus, placental EVs may have the potential to be used as circulating indicators of placental health status.

Preeclampsia (PE) is a pregnancy-specific disorder affecting 3-5% of all pregnancies world-wide. It is a major cause of maternal and neonatal morbidity and mortality. The maternal phenotype of PE is characterized by high blood pressure and damage to another end organ system, which only develops after 20 weeks of gestation. PE has 2 subtypes: early-onset PE (<34 weeks, EOPE) and late-onset PE (≥34 weeks, LOPE)(1). The pathogenesis of these two subtypes are believed to be different. It is hypothesised that EOPE occurs as a result of syncytiotrophoblast stress caused by insufficient trophoblast invasion and inadequate spiral arteray remodeling of maternal spiral arteries in the early stage of pregnancy. LOPE is proposed to result from an interplay between a senescent placenta and maternal predisposition for cardiovascular and metabolic disease. EOPE can cause severe adverse outcomes for both the mother and the fetus, and the rates of adverse maternal and fetal outcomes are severalfold higher in EOPE compared to the ones without EOPE. Early diagnosis of EOPE holds the key to early monitoring and intervention and improving maternal and neonatal prognosis. Studies have shown early intervention (≤16 weeks) with low-dose of aspirin can improve both maternal and neonatal outcomes (25-27). However, predicting PE is challenging: there are no prognostic or diagnostic laboratory assays for PE before 20 weeks of gestation (7).

For both subtypes of PE, the only cure is to deliver the placenta which shows that it is both necessary and sufficient for the disease. It is well established that plasma placental EVs are higher in PE than normal pregnancy (NP), if measured in the second and third trimesters (3, 8, 9). There is also evidence that plasma placental EVs are more abundant in EOPE than LOPE (9, 10). Additionally, placental EVs' protein expression changes in PE. The inventors have previously shown upregulation of placental protein 13, siglec-6, and CD10 (neprilysin), and downregulation of eNOS, on placental EVs in PE compared to NP (11-14). However, these quantitative and qualitative differences in placental EVs in PE have only been observed after the onset of the disease. Placental EVs are detectable in plasma from 6 weeks gestation, but there have been no reports of changes in circulating placental EVs' quantity or phenotype in PE in the first trimester.

### SUMMARY

The inventors have identified a set of placental sEV biomarkers that can distinguish subjects who go on to have a normal pregnancy or develop late onset preeclampsia (LOPE) from those who develop early onset preeclampsia (EOPE). Importantly, patients who go on to develop EOPE can be identified using samples obtained during the first trimester of pregnancy. Hence, the biomarkers identified by the inventors can be used to identify patients at risk of developing EOPE in the first trimester, thereby providing a window of opportunity for vital early intervention.

Accordingly, in a first aspect the invention provides a method for prognosing an increased risk of early onset preeclampsia (EOPE) in a pregnant subject before the disease onset, the method comprising quantifying the detected absolute numbers of small placental extracellular vesicles (sEVs) that express three pairs of biomarkers, wherein the pairs of biomarkers are CD10+CD63+, CD10+placental alkaline phosphatase (PLAP)+, and CD63+PLAP+, in a plasma or serum sample obtained from the subject, and comparing the quantity of CD10+CD63+, CD10+PLAP+ and CD63+PLAP+ sEVs in the sample with reference quantities of CD10+CD63+, CD10+PLAP+, and CD63+PLAP+ sEVs, wherein an increased quantity of CD10+CD63+, CD10+PLAP+ or CD63+PLAP+ sEVs in the sample compared to the reference quantities indicates that the subject has an increased risk of developing EOPE.

The inventors demonstrated the invention using an ExoCounter device. Hence, the CD10+CD63+, CD10+PLAP+ and CD63+PLAP+ sEVs may be quantified using an ExoCounter device.

In a further aspect, the invention provides a method for preventing early onset preeclampsia (EOPE) in a pregnant subject, wherein the method comprises administering prophylactic treatment for EOPE to the subject, and wherein the subject has been prognosed as being at increased risk of EOPE using the method of prognosis.

In a further aspect, the invention provides aspirin for use in a method of preventing early onset preeclampsia (EOPE) in a pregnant subject, wherein the method comprises administering the aspirin to the subject before 16 weeks gestation, and wherein the subject has been prognosed as being at increased risk of EOPE using the method of prognosis.

In a further aspect, the invention provides the use of aspirin in the manufacture of a medicament for preventing early onset preeclampsia (EOPE) in a pregnant subject, wherein the subject has been prognosed as being at increased risk of EOPE using the method of prognosis.

In a further aspect, the invention provides the use of an ExoCounter device in a method of prognosing an increased risk of early onset preeclampsia (EOPE) in a subject before the disease onset, wherein the use comprises (I) incubating a plasma or serum sample obtained from the subject with anti-CD63, anti-CD10 and/or anti-PLAP sEV-capture antibodies, wherein the capture antibodies are coated on the surface of an optical disc comprising a plurality of wells, wherein the base of each well comprises a plurality of grooves, wherein the capture antibodies are coated on the base of the grooves, wherein the incubation conditions are suitable for binding of sEVs in the sample to the capture antibodies in the grooves; (II) washing un-bound sample from the disk; (III) incubating the captured sEVs in the grooves with detection nanobeads, wherein the detection nanobeads are coated with anti-CD63, anti-CD10 or anti-PLAP sEV-detection antibodies, wherein the target antigen (CD63, CD10 or PLAP) of the capture antibodies in a well is different from the target antigen (CD63, CD10 or PLAP) of the detection antibodies coated on the nanobeads incubated in the same well, and wherein the incubation conditions are suitable for binding of detection antibody coated on single nanobeads to single sEVs in the grooves; (IV) washing un-bound nanobeads from the disk; (V) counting the number of nanobeads bound to sEV in the grooves to quantify the detected absolute numbers of CD10+CD63+, CD10+PLAP+ and CD63+PLAP+ sEVs in the sample; (VI) comparing the detected absolute numbers of CD10+CD63+, CD10+PLAP+ and CD63+PLAP+ sEVs of step (V) with reference quantities of CD10+CD63+, CD10+PLAP+ and CD63+PLAP+ sEVs; (VII) determining an increased quantity of CD10+CD63+, CD10+PLAP+ or CD63+PLAP+ sEVs in the sample compared to the respective reference quantities of CD10+CD63+, CD10+PLAP+ or CD63+PLAP+ sEVs; and (VIII) prognosing an increased risk of early onset preeclampsia (EOPE) in the pregnant subject, wherein the increased risk is indicated by the determination in step (VII).

In a further aspect, the invention provides an optical disc wherein the optical disc comprises a plurality of wells for holding a serum or plasma sample, wherein the base of each well comprises a plurality of grooves, wherein the base of the grooves are about 160 nm in width and the top of the grooves are about 260 nm in width, wherein the base of the grooves in a well of the disc is coated with anti-CD10 antibodies and wherein the base of the grooves in a different well of the disc is coated with anti-CD63 antibodies.

In a further aspect, the invention provides a kit comprising the optical disc above and two sets of light-reflective nanobeads, wherein the nanobeads of one of the two sets are coated with anti-placental alkaline phosphatase (PLAP) antibodies and the nanobeads of the other set are coated with anti-CD63 antibodies.

The invention will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the invention which is defined by the claims. All documents cited herein, whether supra or infra, are expressly incorporated by reference in their entirety.

The present disclosure includes the combination of the aspects and features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “an antibody” includes two or more such entities. In general, the term “comprising” is intended to mean including but not limited to. In some embodiments of the invention, the word “comprising” is replaced with the phrase “consisting of” or the phrase “consisting essentially of”. The term “consisting of” is intended to be limiting. The term “consisting essentially of” should be understood to mean that the sequence comprises no additional sequence units or elements that materially affect the function of the sequence element. The term “about” typically means+/−20% or +/−10%.

Section headings are used herein for convenience only and are not to be construed as limiting in any way.

### DESCRIPTION OF THE FIGURES

FIG. 1. A typical dics-like solid support. A) Plan view of an optical disc showing concentric arrangement of grooves. A spiral arrangement may also be used and allows for continuous scanning using a laser diode or the like. A small number of concentric grooves is shown for illustrative purposes. However, a typical support would include many more rings or spiral turns, more narrowly spaced together. An overlaid plate provides wells on one surface of the disc. Inset shows cross-section of well 3 with grooves coated with capture antibody. B) The principle for exosome capture and quantification by ExoCounter. The disc is coated with the capture antibody and then the samples are directly applied to the disc. The target antibody conjugated nanobeads is then added. Beads that specifically bound to exosomes remained after washing step and bead and exosome immobilised on the disc is at one-to-one ratio. The disc was then inserted into a disc-reader where each circular trenches were scanned by a laser in an orderly manner. Each bead will modify a light refraction upon laser excitation. The light refraction signal is captured by a photodetector and converted to pulses. The number of pulses correspond to the number of beads.

FIG. 2. (A) psEVs characterized by western blot. PL indicates placenta lysate. HEK293 lysate as negative controls for PLAP and positive control for cytochrome C. The characterization markers were Alix (95 kDa), CD63 (30-65 kDa), PLAP (66 kDa), Syntenin (30 kDa), CD9 (23 kDa), Cytochrome c (12 kDa). (B) Representative pictures of psEVs characterized by TEM of higher magnification. Scale bar=1 μm. (C) Representative pictures of psEVs characterized by TEM at lower magnification. Scale bar=5 μm. (D) Representative characterization of psEVs by NTA.

FIG. 3. ExoCounter assay optimisation. PBS or ACE was tested as coating antibody diluent, 1% or 0.01% Casein in PBST was tested as blocking buffer (A). Assay reproducibility and linearity with the application of identified optimal assay conditions on different dates in the same organisation (B). Assay reproducibility and linearity with the application of identified optimal assay conditions on different dates in different organisations (C). PLAP was used as both coating antibody and bead antibody for psEVs detection. The amount of protein tested were from 0, 0.03, 0.1, 0.3, 1 and 3 (μg) in Figure (A&B), from 0.001, 0.4, 0.2, and 1 (μg) were tested in (C). For all figures, the x-axis represents the amount of protein tested (μg), the y-axis represents PLAP-PLAP psEVs counts detected by ExoCounter with background (A) or after background correction (B&C). PBS as coating antibody diluent and 0.01% Casein in PBST as blocking buffer for both (B) and (C). n=3 for all data points. The error bar of each point was calculated by the standard deviation (SD) and some error bars were invisible due to the size of the error bars being smaller than the symbols.

FIG. 4. (A) The association of the three tetraspanins CD9, CD63 and CD81 with PLAP on psEVs detected by MACSPlex. Y-axis indicates the geometric mean fluorescent intensity of all the samples measured, n=4. (B) The quantification of psEVs isolated from placenta perfusion using PLAP-CD9 and PLAP-CD63 by ExoCounter. The correlation coefficient between PLAP-CD63 and PLAP-CD9 is calculated by the covariance of sEVs counts at each amount of protein tested for PLAP-CD63 and PLAP-CD9, divided by the product of their standard deviations. (C) The quantification of psEVs isolated from placenta perfusion using CD63-PLAP, PLAP-CD63, CD63-CD63 and PLAP-PLAP. The x axis represents the amount of protein (μg) of psEVs tested which includes 0.03, 0.1, 0.3, 1 and 3 μg of psEVs. The y axis represents the absolute counts of psEVs detected by ExoCounter after background correction. The error bar of each point was calculated by the SD of the triplicates and some error bars were invisible due to the size of the error bars being smaller than the symbols.

FIG. 5. The association of each exosomal or cell lineage markers on psEVs with PLAP detected by MACSPlex. Each bar corresponds to sEVs captured by antibodies specified which have a co-expression of PLAP. Y-axis indicates the geometric mean fluorescent intensity of all the samples measured, n=4.

FIG. 6. The CD63-PLAP psEVs counts of serum and plasma from the third trimester of the same NP donor (n=7) detected by ExoCounter. The x-axis is the sEVs/mL in serum and the y-axis is the sEVs/ml in the plasma. The linear regression formula is y=0.86x+431.6 with R² equal to 0.9113.

FIG. 7. CD10-PLAP, CD10-CD63 and CD63-PLAP psEVs counts in peripheral vein (PV) and paired uterine vein (UV) from healthy pregnant women detected by ExoCounter, n=7 (A). Comparison of counts of CD10-PLAP (B), CD10-CD63 (C) and CD63-PLAP (D) sEVs in NP, WWD-EOPE and WWD-LOPE plasma samples from the first, second and third trimester. Each point represents the mean level of the group and the error bars were calculated by the standard deviation (SD). The y-axis represents the psEVs counts/ml after background (BG) correction.

FIG. 8. A) Counts of CD10-PLAP, CD10-CD63, and CD63-PLAP psEVs in NP and WWD-EOPE serum samples from the first trimester. (B) Counts of CD10-PLAP, CD10-CD63, and CD63-PLAP psEVs in NP and WWD-LOPE serum samples from the first trimester. For both figures, each point represents a sample point and the counts of CD10-PLAP is represented by circle dot, CD10-CD63 is represented by square dots and CD63-PLAP is represented by the triangle dots. The red line represents the median level of the group and ** denotes P≤0.01. The statistic test for both figures was carried using the Mann-Whitney test. The y axis represents the sEVs counts after background (BG) correction.

FIG. 9. Effect of casein concentration in beads dilution buffer on non-specific signal (IgG1-CD63) (A), specific signal (CD63-CD63) from plasma samples (B) and signal to noise ratio (S/N ratio (C). Effect of PBS or PBST as plasma sample diluent on non-specific signal (IgG1-CD63) and specific signal (CD10-CD63) on spiked and non-spiked plasma samples (D). 4 mg/ml psEVs isolated from placenta perfusion was spiked into plasma samples from healthy non-pregnant donors as placental specific signals.

FIG. 10. Experimental design and flow chart.

### DETAILED DESCRIPTION

Extracellular vesicles (EVs) are cell-derived membrane-bound particles laden with proteins, lipids and nucleic acids. The change of quantity and characteristics of EVs present in biofluids might be responsible for the pathology of diseases. The inventors have developed an assay to quantify and characterise placental small EVs using placental sEVs (psEVs), firstly using psEVs isolated from perfused placentas and subsequently from pregnant plasma and serum. Both ExoCounter and MACSPlex showed CD63, but not CD9 or CD81, to be the major tetraspanin molecule co-expressed with placental alkaline phosphatase (PLAP). To investigate disease- and gestational age-specific changes, the inventors analysed psEV counts in maternal plasma samples taken at each of the three trimesters from normal pregnancy (NP, n=3), women who developed early-onset preeclampsia (WWD-EOPE, n=3) and women who developed late-onset preeclampsia (WWD-LOPE, n=4) patients. Using three antibody pairs, CD10-PLAP, CD10-CD63 and CD63-PLAP, psEVs counts for either NP, WWD-EOPE or WWD-LOPE remained similar for all three trimesters (i.e. no gestational changes either in disease or normal status, the counts from first trimester were similar to the second and third trimester in NP, WWD-EOPE and WWD-LOPE). However, higher psEVs counts of all three antibody pairs were detected in WWD-EOPE compared to both NP and WWD-LOPE as early as the first trimester. The inventors further validated these findings by a larger sample size of first-trimester serum samples among NP (n=10), WWD-EOPE (n=8) and WWD-LOPE (n=9). Significantly higher CD10-PLAP (p<0.01) and CD63-PLAP (p<0.01) psEVs counts were found in WWD-EOPE compared to NP. Hence, the assay described herein can identify patients at risk of developing EOPE at the first trimester, thereby providing a window of opportunity for early intervention.

### Method/Assay

The invention provides a method/assay for diagnosing/prognosing/determining an increased risk of early onset preeclampsia (EOPE). The increased risk is typically determined before disease onset; i.e. before symptoms arise or before health impacts arise for the mother and/or the fetus. Typically the method is carried out in the first trimester of pregnancy in the subject, or within about the first 12 or 16 weeks of pregnancy, or between about 10 and about 12 weeks (about 70 to 84 days) of pregnancy. This is advantageous because early intervention opens up different treatment options, e.g. administration of aspirin, and improves outcomes.

The method comprises quantifying the small extracellular vesicles (sEVs) having particular biomarker pairs or combinations in a sample that has been obtained from the subject. Typically the sample is a serum or plasma sample. The sample may be a different bodily fluid such as a urine, saliva or cerebral spinal fluid sample. The biomarkers most typically comprise or consist of CD-10 (also known as neprilysin) and PLAP. The biomarkers may comprise or consist of CD-63 and PLAP; or CD-10 and CD63. The biomarker pairs may be any two of CD-10 and PLAP, CD-63 and PLAP, and CD-10 and CD63. Most typically, all three biomarkers are used to quantify the placental sEVs. Most typically placental sEVs expressing all three possible pairs of these three biomarkers are quantified, i.e. sEVs expressing both CD-10 and PLAP, sEVs expressing both CD-63 and PLAP, and sEVs expressing CD10 and CD63. The quantity of sEVs expressing each pair of biomarkers may be determined separately.

The quantification may involve capturing sEVs using antibodies against one of the, or one of each pair, of biomarkers, and detecting the sEVs using a second antibody against the other of the, or each pair of, biomarkers. Hence, the method uses one or more pairs of a sEV capture antibody and a sEV detection antibody (also referred to herein as a labelling or conjugated antibody), wherein the capture antibody and the detection antibody of each pair have different target biomarker antigens. For example, the method may use pairs of sEV-capture:sEV-detection antibodies comprising anti-CD10:anti-CD63, anti-CD10:anti-PLAP and/or anti-CD63:anti-PLAP antibodies. However, one or more of these configurations of sEV-capture:sEV-detection antibodies may also be reversed, i.e. pairs comprising anti-CD63:anti-CD10, anti-PLAP:anti-CD10 and/or anti-PLAP:anti-D63 antibodies may be used.

Hence, the method may comprise capturing sEVs from the sample using sEV capture antibodies against (i) CD10 or CD63, (ii) CD10 or PLAP, and (iii) CD63 or PLAP; and incubating sEVs from the sample with sEV detection antibodies against the other biomarker of each pair (i) CD10 or CD63, (ii) CD10 or PLAP, and (iii) CD63 or PLAP, respectively; and detecting the detection antibodies bound to captured sEVs. The capture and incubation steps may be performed in either order or simultaneously. The detected detection antibodies indicates the quantity of sEVs in the sample that express each pair of biomarkers. In a more specific case, the method may comprise capturing sEVs in the sample using sEV capture antibodies against (i) CD10 or CD63, (ii) CD10 or PLAP, and (iii) CD63 or PLAP; incubating the sEVs captured by antibodies (i), (ii) and (iii) of step (I) with sEV detection antibodies against the other biomarker of each pair (i) CD10 or CD63, (ii) CD10 or PLAP, and (iii) CD63 or PLAP, respectively, and detecting the detection antibodies bound to the captured sEVs. The detection antibodies may be bound to a detectable moiety. Hence, the method comprises detecting the detectable moieties bound to captured sEVs to quantify the sEVs in the sample that express the or each pair of biomarkers. In some cases, the capture antibodies may be (i) anti-CD10, (ii) anti-CD10, and (iii) anti-CD63 antibodies; and the detection antibodies may be (i) anti-CD63, (ii) anti-PLAP, and (iii) anti-PLAP antibodies, respectively.

The capture antibodies may be immobilised on a solid support. In some cases, sample may be mixed with detection antibody before adding to the solid support for capture on the capture antibodies. In other cases, a solution comprising the detection antibodies (with conjugated detectable moiety) may be applied to the solid support first and then the sample. Hence, in some cases, the method may comprise (I) incubating the plasma or serum sample obtained from the subject with the anti-CD63, anti-CD10 and/or anti-PLAP sEV capture antibodies and the detection antibodies bound to a detectable moiety, wherein the capture antibodies are immobilised on a solid support, and wherein the incubation conditions are suitable for binding of sEVs in the sample to both the capture antibodies and the detection antibodies; (II) washing unbound sample and detection antibody from the solid support; and (III) detecting the detectable moieties bound to the solid support via a detection antibody, sEV and capture antibody, to quantify the sEVs in the sample that express each pair of biomarkers.

In more typical cases, the sample is applied to the solid support for capture of sEVs in the sample to capture antibodies coating the surface of the solid support first. Un-bound sample/sEVs may be washed from the solid support before applying the detection antibodies. This washing step removes nonspecific molecules other than target sEVs to suppress background noise.

The method may comprise incubating the capture antibodies on the solid support with the sample under conditions suitable for binding of sEVs in the sample (and expressing the target antigen of the capture antibody) to the capture antibody. The method may further comprise washing unbound sample from the solid support. The method further comprises incubating the captured sEVs with detection antibodies (e.g. bound to a detectable moiety) under conditions suitable for binding of the detection antibody to captured sEVs (that express the target antigen of the detection antibody). The method may further comprise washing un-bound detection antibody from the solid support. The method further comprises detecting the detection antibodies bound to the solid support (via captured sEVs and capture antibodies immobilised on the solid support), e.g. by detecting the detectable moieties bound to the solid support (via the detection antibody, sEVs and capture antibodies), to quantify sEVs (that express the target antigens of both the capture antibodies and the detection antibodies) in the sample.

Typically each quantification of sEVs expressing a particular pair of biomarkers is carried out on a different region or area of the solid support, e.g. in separate wells. Hence, only one type of capture antibody (anti-CD10, anti-CD63 or anti-PLAP) is bound to the solid support in each region/well. The sample may be divided between the regions or wells. Alternatively, a separate solid support may be used for each pair of biomarkers.

The method may further comprise a step of removing larger EVs (typically EVs >about 200 nm; otherwise EVs >200, or >about 190, or >about 210 nm) from the sample, e.g. before applying the sample to the solid support. Large EVs may be removed, for example, by filtering/using a filter. Alternatively, centrifugation could be used. For example, centrifugation at 10,000 g or more can precipitate lager EV in the sample. The rest of the sample can then be recovered without the larger EVs. Alternatively, larger EVs may be excluded from binding to the capture antibodies (and/or the detection antibodies), or may be prevented from accessing or binding to the capture antibodies (and/or the detection antibodies) in a particular region or regions of the solid support where the detection moieties will be detected (as discussed further below). For example the size and shape of the solid support may be such as to prevent binding of larger EVs to the capture antibodies (and/or to the detection antibodies).

Typically only one detection moiety binds to each captured sEV. The method only allows binding of one detection moiety per captured sEV. The method essentially prevents binding of multiple, i.e. more than one detection moiety (via detection antibodies) to a single sEVs. This can improve the accuracy of the quantification because single counts of detectable moiety (bound to the detection antibodies) directly (or essentially) correlates to single sEVs. This may be achieved by limiting access or binding of more than one detection moiety (via detection antibodies) to single captured sEVs. For example, the size and shape of the solid support and/or steric hindrance between multiple detection moieties in the vicinity of a captured sEV may prevent or substantially reduce the incidence of binding by multiple detection moieties. For example, binding of single sEVs by multiple detection moieties may be reduced by at least 80%, or by at least 85%, 90%, 95%, 97%, 98%, or 99%. Again, this may apply only to sEVs captured in a particular region or regions of the solid support where the detection moieties will be detected, as discussed further below. Additional (s)EVs/detection antibodies/detection moieties bound directly or indirectly to the solid support outside of these specific region(s) may either not be detected (e.g. because these other regions are not scanned for detection moiety) and/or are not counted in/are excluded from the quantification.

In some cases, for example, the surface of the solid support may include grooves or other recesses having a size and shape that permits binding of capture antibodies and binding of sEVs to the capture antibodies within the grooves or recesses, but that excludes larger EVs and/or detection antibodies bound to detection moiety, i.e. because the EVs and/or detection moiety (or detection moiety+bound detection antibody) are too large or bulky to enter or to fully enter the groove(s)/recess(es). Detection antibody can still bind to sEVs captured within the grooves/recesses. However, the solid support/solid support surface may have a shape that prevents more than one side of a detection moiety coated with detection antibody, or more that one detection moiety bound to detection antibody, from accessing sEVs captured in the grooves/recesses. Hence, one detection moiety bound to the solid support (via a captured sEV and a capture antibody in the groove or recess) essentially corresponds to one captured sEV.

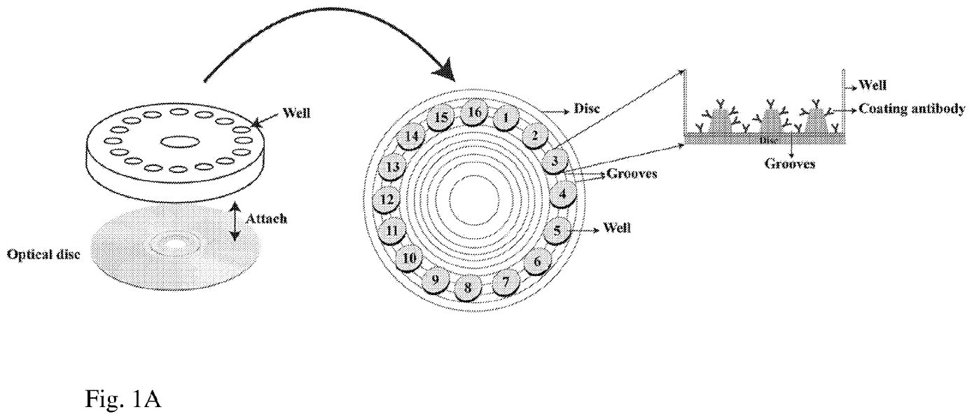

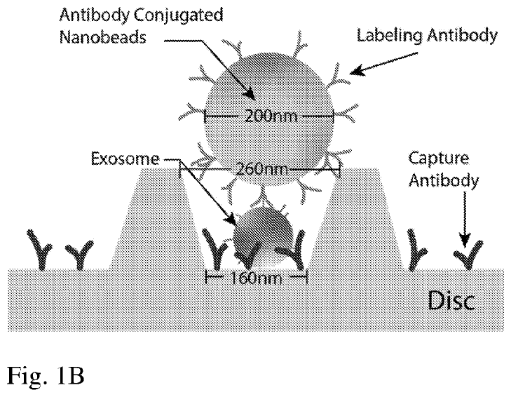

Placental sEV typically have a distribution of sizes between about 100 and about 220 nm in diameter. The size and shape of the grooves/recesses is such as to allow entry of sEVs within all or part of this size distribution. Hence, in some cases a groove/recess may have a smallest opening/diameter of at least about 150 nm, or at least about 160 nm in diameter. To exclude larger EVs, the smallest width/opening/diameter, for example at the bottom of a groove or recess, may be no more than (about) 200 nm or 190 nm or 180 nm or 170 nm or 165 nm or 160 nm across. The largest opening or diameter of a groove or recess may be smaller than the smallest diameter of the detectable moiety with bound detection antibody. In a typical embodiment, the largest width, opening into or diameter of a groove or recess, for example at the top of the groove where the sEVs enter, may be about 240 to 280 nm, or about 250 to 270 nm, or about 255 to 265 nm, or about 260 nm across/in width. The grooves or recesses are typically deep enough to allow sEVs to enter the groove or recess. Hence, the depth is typically at least about 50 nm, or at least about 60 nm. In some cases, the depth may be at least about 70 nm, 80 nm, 90 nm or 100 nm. In some cases the depth may be up to about 80 nm, or about 90 nm, 100 nm or 110 nm. Typically, the depth of the grooves is about 60 to 70 nm. In a particular case, the solid support comprises a surface having one or more grooves (or recesses) that are about 150 to 170 nm, or about 155 to about 165 nm, or about 160 nm in width at the base of the groove (or recess) and about 260 nm in width at the top of the groove (FIG. 1B). This configuration allows sEVs to enter the groove/recess and bind to capture antibodies coated on the surface of the solid support within the groove, and is suitable for use with detection nanobeads of about 200 nm in diameter and coated with detection antibodies. Such detection nanobeads with bound detection antibodies are too large to fully enter the grooves, but the detection antibodies on one side of the surface of the nanobeads are able to bind to sEVs captured on the capture antibodies within the grooves. This configuration also prevents multiple detection nanobeads from binding, via the detection antibodies, to single sEVs within a groove.

Typically the regions outside of and/or between the grooves or recesses on the surface of the solid support (which may be referred to herein as protruding regions or protruding portions of the solid support surface) are narrow compared to the typical diameter of the detectable moiety bound to detection antibody(ies). This feature (further) inhibits binding of multiple detection moieties (via detection antibodies) to single sEVs captured in the grooves or recesses. In some cases, the protruding portions may have a narrowest width of less than about 250 nm, or less than about 200 nm, 150 nm, 100 nm, or 80 nm. Most typically the protruding portions have a width of about 60 nm.

In a particularly convenient configuration, the solid support may have a circular disc shape or be a typical optical disc, as known to those in the art. The solid support may be engraved circularly on one face with a groove of spiral form. In a specific example, the periodic pitch of the spiral is about 320 nm. In another convenient configuration, the solid support may be a disc engraved on one face with concentric circular grooves. Such a disc is shown in FIG. 1A. The dimensions of the groove may be as set out above.

The detectable moiety may be a light-reflective (or diffractive) moiety and/or a nanobead. A nanobead is approximately spherical or sphere-like. A typical nanobead used in the method of the invention may be about 180 to 220 nm, or about 190 to 210 nm or about 195 to 205 nm or about 200 nm in diameter. The nanobead may be both light-reflective/diffractive and magnetic. For example, the nanobead may comprise one or more magnetic materials or ferrites, for example the nanobeads may comprise a ferrite core. The magnetic materials (ferrites) enhance the reflectivity of nanobeads and the diffraction generated with nanobeads. Using magnetic beads can also assist with handling and directing the beads using a magnetic field. The nanobeads may have a resin or polymer coat, such as a coat of polyGMA (glycidyl methacrylate). The detection antibodies may be coated on the surface of the nanobead. This may be done using methods known to those skilled in the art.

Light-reflective/refractive moieties may be illuminated, for example with a laser spot or using a laser diode. Light reflected/refracted from the solid support/disc is modified by the presence of a light reflective/refractive moiety bound (via a detection antibody, sEV and capture antibody) to the solid support. Hence, the presence of bound detection moiety, and hence sEV (expressing the relevant biomarkers) may be detected. For example a photodetector may be used to record the light reflected/refracted from the solid support/disc/grooves and/or to detect the presence of bound detectable/light-reflective/refractive moiety. For example, the light refraction signal may be captured by a photodetector and converted to pulses. The number of pulses correspond to the number of detection moieties/nanobeads. Where there is a 1:1 ratio of bound detection moieties/nanobeads:bound sEVs, as described herein, the number of pulses corresponds to the number of sEVs. In the reflection of light, the diffraction by nanobeads modifies reflectivity, more precisely the reflectivity decreases, and the presence of nanobeads may be detected from this reflectivity change.

The laser or other light source can be programmed to illuminate the surface of the solid support in a tightly controlled spatial and/or temporal pattern. The light source may be turned on and off as different regions of the surface are illuminated. However, it is convenient to use a solid support having a continuous groove in the surface so that the laser/light-source can scan continuously along the or a length of the groove. As the light/laser spot moves along the groove, detectable moieties bound via detection antibody to captured sEVs within the groove are detected, as described above. For example, a convenient configuration is a disc-shaped solid support or optical disc comprising a spiral groove in one surface. Another convenient configuration is a set of concentric circular grooves in one surface of a disc-like solid support or optical disc. The light/laser spot can scan along the spiral groove from the inner to the outer (or outer to inner) side of the disc surface. Light reflected from the surface is recorded. The path of the light/laser spot/illumination may correspond to the positions on the solid support surface where only single light-reflective moieties can bind to single sEVs. For example, the light/laser source may be programmed only to illuminate the recesses or grooves in the surface. Hence single detected light-reflecting moieties (e.g. nanobeads) correspond to single sEVs that express the target antigens of both the capture antibody and the detection antibody bound to the detected light-reflecting moieties.

Other known detection moieties may be used and detected in a similar way. For example fluorescent moieties/nanobeads/dyes or quantum dot moieties/nanobeads may be used and detected using suitable detection devices as are known to those in the art.

The method described above quantifies the detected absolute numbers of sEVs having the relevant biomarkers in the sample. Not every relevant sEV in the sample is counted because some sEVs in the sample will be washed away during the assay. Others may bind to capture antibody outside of the detection regions, e.g. outside of the grooves/recesses. Hence, the detected absolute numbers are quantified. This differs from other current methods of detecting EVs, which typically use a relative quantification and is advantageous because it is less effected by variation in other factors. Assays that quantify sEVs lose some true signals from doing the experiments. However, by doing serial dilution for sample input, it may be possible to detect the serial dilution effect by the counts (outcomes). For example, using an Exocounter device it is possible to generate a R square over 0.99 for the linear regression on sample concentration tested against the counts within the linear quantifiable range. Other relative quantification methods can not achieve this clear serial dilution effect.

The solid support may comprise a plurality of wells or otherwise defined and separated areas that prevent mixing between sample or reagents and the like added to different wells/areas. Each well/area may have the same shape, size and/or number or length/area of grooves/recesses as described herein. In one embodiment, the wells may be defined by laying/attaching a plate comprising a plurality of openings on top of the surface of a solid support, e.g. an optical disc, comprising the grooves or recesses. The area of the solid support under each opening forms the base of each well and the internal walls of each opening in the plate form the walls of each well. In this case, the solid support comprising the grooves/recesses and the plate together may form the “solid support” or “optical disc” as referred to herein. An example of this is shown in FIG. 1A.

Including multiple wells allows multiple samples, multiple measurements from the same divided sample, and/or multiple combinations of capture and detection biomarkers to be interogated using the same support and/or in the same experiment. For example, the support may include at least one well coated with/having grooves coated with anti-CD63 capture antibody, and two wells coated with/having grooves coated with anti-CD10 capture antibody, or multiples thereof, e.g. at least two with anti-CD63 and four with anti-CD10, or at least three with anti-CD63 and six with anti-CD10 and so on. Control wells may also be included. For example, one control well may be used per pair of capture and detection antibodies, wherein the control well receives no capture antibody/PBS only. Sample may be equally divided between the wells/areas and sEVs in the sample are allowed to bind to the capture antibodies in each well/area. Un-bound sample is washed away. Detection antibodies (bound to detection moieties) are then added to the wells, wherein the target antigen of the detection antibodies added to each well is different from the target antigen of the capture antibody in the same well. Hence, each well is used to detect sEVs that express a particular pair of biomarkers corresponding to the target antigens of the capture and detection antibodies used in each respective well. In one example, anti-PLAP detection antibody may be added to a well coated with anti-CD63 capture antibodies; anti-PLAP detection antibody may be added to a well coated with anti-CD10 capture antibodies; and anti-CD63 detection antibody may be added to a well coated with anti-CD10 capture antibodies. Using this or similar configurations as described herein, all three of CD10+CD63+, CD10+PLAP+ and CD63+PLAP+ sEVs in the sample may be quantified using a single solid support.

In one embodiment, the method/assay of the invention may comprise incubating the sample obtained from the subject with anti-CD63, anti-CD10 and/or anti-PLAP sEV-capture antibodies, wherein the capture antibodies are coated on the surface of a solid support (e.g. an optical disc, as described herein) comprising a plurality of wells, wherein the base of each well comprises a plurality of grooves or recesses, wherein the capture antibodies are bound/coated within and/or on the base of the grooves or recesses, wherein each of the wells is coated with one of the sEV capture antibodies, anti-CD63, anti-CD10 or anti-PLAP, and wherein the incubation conditions are suitable for binding of sEVs in the sample to the capture antibodies in the grooves or recesses. The method may further comprise washing un-bound sample from the solid support. The method further comprises incubating the captured sEVs in the grooves/recesses/wells with detection moieties such as nanobeads, wherein the detection moieties or nanobeads are bound to/coated with anti-CD63, anti-CD10 or anti-PLAP sEV-detection antibodies, wherein the target antigen (CD63, CD10 or PLAP) of the capture antibodies in a well is different from the target antigen (CD63, CD10 or PLAP) of the sEV-detection antibodies incubated in the same well, and wherein the incubation conditions are suitable for binding of detection antibody bound to/coated on single detectable moieties/nanobeads to single sEVs in the grooves or recesses. The method may further comprise washing un-bound nanobeads from the solid support. The method may further comprise counting the number of detectable moieties/nanobeads bound to sEV in the grooves or recesses. These counts may be used to quantify the sEVs in the sample that express the pair of biomarkers targeted by the capture:detecton antibody pair. This method may be used to quantify the sEVs having any one, or any combination of the biomarker pairs: CD63 and CD10, CD63 and PLAP, and CD10 and PLAP.

Unlike other current EV analysis platforms, such as flow cytometry, the method of the invention may be carried out using minimal sample pre-processing and preparation steps. This significantly reduces pre-analytical variation and is ideal for use in clinical laboratories. The method of the invention may be carried out using an ExoCounter device. The solid supports are optical discs that are suitable for use with the ExoCounter device. The ExoCounter is a novel semi-automatic EV quantification assay, which allows the investigator to quantify sEVs by capture on an optical disc with 160 nm grooves utilizing a combination of up to two different antibodies for sEVs adherence and detection (15). The ExoCounter device is described in WO 2014/168020 A1. The features of the device described therein, particularly in the claims therein, may be features of the device used in the methods and uses of the invention described herein. Use of this semi-automatic device is fast and efficient and reduces variability in sample handling.

The inventors have tried various buffers with the ExoCounter assay according to the invention. In some cases the method uses about 0.1% casein in phosphate-buffered saline containing 0.05% Tween-20 (PBST) as a nanobead-detection antibody dilution buffer for the incubation with the captured sEVs.

In some cases, the method comprises incubating the disc grooves comprising the capture antibodies with blocking buffer before incubation with the plasma or serum sample. This step reduces or prevents non-specific binding. The blocking buffer may be about 0.1% casein in phosphate-buffered saline containing 0.05% Tween-20 (PBST).

In some cases, the method comprises the step of immobilising or coating the capture antibodies on the solid support, e.g. an optical disc. In such cases the method may use phosphate-buffered saline (PBS)) as a dilution buffer for coating the capture antibodies on the optical disc. Typically the disc may be coated by the antibody through hydrophobic interactions between the solid support and non-polar residues on the antibody proteins (also known as passive adsorption). Suitable methods of coating the capture antibodies onto the solid support or optical disc are known in the art. Typically just the area on the surface of the support within the relevant well(s)/regions to which subject or control samples are to be added are coated with a particular capture antibody. Typically the whole surface of the support within the relevant well is coated with the capture antibody, including the base of any grooves or recesses as described herein.

The method may further comprise washing unbound capture antibodies from the support, i.e. before incubation with blocking buffer or sample.

### Antigen-Binding Molecules and Antibodies

The term ‘antibody’ as used herein may relate to whole, full-length antibodies (i.e. comprising two antibody heavy chains and two light chains inter-connected by disulphide bonds), as well as antigen-binding fragments thereof. The term may also encompass VHH antibodies (heavy-chain antibodies), having two heavy chains only and no light chains, and antigen binding fragments thereof. Antibodies typically comprise immunologically active portions of immunoglobulin (Ig) molecules, i.e., molecules that contain an antigen binding site that specifically binds (immunoreacts with) an antigen. By “specifically binds” or “immunoreacts with” is meant that the antibody reacts with one or more antigenic determinants of an antigen, i.e. in the normal way that antibodies bind to antigen. Each heavy chain is typically comprised of a heavy chain variable region (abbreviated herein as HCVR or VH) and at least one heavy chain constant region. Each light chain is typically comprised of a light chain variable region (abbreviated herein as LCVR or VL) and a light chain constant region. Typically the variable regions of the heavy and light chains contain a binding domain that interacts with an antigen, except for VHH antibodies where the variable regions of the heavy chains only comprise the antigen binding domain. The VH and VL regions can be further subdivided into regions of hypervariability, termed complementarity determining regions (CDR), interspersed with regions that are more conserved, termed framework regions (FR). Antibodies may include, but are not limited to, polyclonal, monoclonal, chimeric, recombinant, dAb (domain antibody), single chain, Fab (fragment antigen-binding regions), Fab′ and F(ab′)2 fragments, scFvs (single chain variable fragments), and Fab expression libraries.

The antibody used in the invention may be an antigen-binding fragment. An antigen-binding fragment used in the invention binds to the same epitope as a reference full length antibody or parent antibody, i.e. the antibody from which the antigen-binding fragment is derived. An antigen-binding fragment typically retains the parts of the parent antibody that interact with the epitope. Typically, the antigen-binding fragment retains the same or similar binding affinity to the antigen as the reference antibody. Methods for creating and manufacturing antibody fragments are well known in the art (see for example Verma R et al., 1998, J. Immunol. Methods, 216, 165-181).

An antibody used in the invention may be a monoclonal antibody. Monoclonal antibodies (mAbs) may be produced by a variety of techniques, including conventional monoclonal antibody methodology, for example those disclosed in “Monoclonal Antibodies: a manual of techniques” (Zola H, 1987, CRC Press) and in “Monoclonal Hybridoma Antibodies: techniques and applications” (Hurrell J G R, 1982 CRC Press). Antibodies used in the invention may be isolated antibodies. An isolated antibody is an antibody which is substantially free of other antibodies having different antigenic specificities.

The antibodies described herein for use with the invention could also be replaced with other antigen-binding molecules having similar antigen-binding specificity and affinity as antibodies, such as antibody mimetics or aptamers. References to “antibodies” in the detailed description should be read throughout as including such alternative antigen-binding molecules.

Exemplary antibodies used in the Examples described below and that may be used in the invention described herein are provided in Table 1.

TABLE 1
The information of the antibodies used.

| | | Catalog | | Assay |
|---|---|---|---|---|
| Supplier | Description | Number | Clone | Concentration |
| Miltenyi | CD10 | 130-108-025 | 97C5 | 5 μg/mL |
| Biotec | antibody | | | |
| | (neprilysin) | | | |
| Biolegend | LEAFTM | 353014 | H5C6 | 5 μg/mL |
| | Purified | | | |
| | anti-human | | | |
| | CD63 | | | |
| Biolegend | Purified | 312102 | HI9a | 5 μg/mL |
| | anti-human | | | |
| | CD9 | | | |
| In-house | Anti-human | N/A | NDOG2 | 5 μg/mL |
| | placental | | | |
| | alkaline | | | |
| | phosphatase | | | |
| | (PLAP) | | | |
| BioLegend | IgG1 | 400101 | MOPC-21 | 5 μg/mL |

N/A denotes not applicable.

### Optical Disks

In some embodiments the invention provides an optical disc or disc-shaped solid support. The optical disc is (suitable) for use in a method for prognosing/diagnosing/detecting an increased risk of early onset preeclampsia (EOPE) in a pregnant subject before the disease onset in accordance with the invention. The disc may be (suitable) for use with an Exocounter. The disc may have any of the physical features described herein and/or above. For example, (one surface of) the optical disc may comprise a plurality of wells for holding a sample obtained from the subject, as described herein. The base of each well may comprise a plurality of grooves. The base of the grooves may be about about 150 to 170 nm, or about 155 to about 165 nm, or about 160 nm in width at the base of the groove. The top of the grooves may be about 240 to 280 nm, or about 250 to 270 nm, or about 255 to 265 nm, or about 260 nm in width. The side of the disc comprising the wells may comprise a spiral groove. The groove extends from an inner portion of the disc surface to an outer portion of the disc surface. The groove passes through wells of the disc, providing the well grooves. Typically each well has the same configuration and/or length of groove. The groove typically has a consistent size and shape substantially along its length.

The base of the grooves in a well of the disc is coated with antibodies. Typically the antibodies are coated on the whole surface of the disc inside a well, including the base of the grooves of the well. The target of the antibodies coated in the wells is CD10, CD63 and/or PLAP. The antibodies in each well typically target one of these three target antigens. However, different antibodies may be coated on the disc in different wells. The antibodies coated in the wells are anti-CD10, anti-CD63 and/or anti-PLAP antibodies. In some embodiments at least one (or at least two) wells/the grooves of at least one (or at least two) wells are coated with anti-CD10 antibodies and at least one well/the grooves of at least one well are coated with anti-CD63 antibodies. In other cases, anti-CD10 and anti-PLAP antibodies are used, or anti-CD63 and anti-PLAP antibodies are used.

### Kits

In some embodiments the invention provides kits. The kits are (suitable) for use in a method for prognosing/diagnosing/detecting an increased risk of early onset preeclampsia (EOPE) in a pregnant subject before the disease onset in accordance with the invention.

The kit may comprise an optical disc or disc-shaped solid support as described herein. The kit may further comprise sEV detection antibodies as described herein. The antibodies are or include anti-CD10, anti-CD63 and/or anti-PLAP antibodies. In one embodiment the kit includes anti-PLAP antibodies and anti-CD63 antibodies. In other cases, the kit may comprise anti-CD1 and anti-CD63 antibodies, or the kit may comprise anti-PLAP and anti-CD10 antibodies. The sEV detection antibodies of the kit target a different antigen from at least one antibody coated in the wells of the disc. In one particular embodiment, the wells of the disc(s) of the kit are coated with anti-CD10 antibodies, and the detection antibodies of the kit are anti-PLAP antibodies. In another particular embodiment, the wells of the disc(s) of the kit are coated with anti-CD10 and anti-CD63 antibodies, and the detection antibodies of the kit are anti-PLAP and anti-CD63 antibodies. Most typically, the selection of antibodies coated on the disc in the wells and the detection antibodies is such that sEVs that express all three possible pairs of the three biomarkers CD10, CD63 and PLAP can be detected using the kits of the invention in a method or assay according to the invention. The detection antibodies are bound to a detectable moiety. Typically, the detectable moiety is a nanobead as described herein and/or above. In some cases, the kit comprises nanobeads coated with detection antibodies against one of the biomarkers, CD10, CD63 or PLAP. In a particular embodiment, the kit comprises two sets of nanobeads. In one of the two sets of nanobeads, the nanobeads are coated with anti-CD63 antibodies. In the other set of nanobeads, the nanobeads are coated with anti-PLAP antibodies. The nanobeads may have any of the features described herein and above. The nanobeads may be light-reflective nanobeads or magnetic light-reflective nanobeads. The nanobeads may comprise ferrites or a (mixed) ferrite core. The nanobeads may further comprise a resin coat, for example a coat of glycidyl methacrylate (polyGMA). The nanobeads are typically about 180 to 220 nm, or about 190 to 210 nm or about 195 to 205 nm or about 200 nm in diameter.

The kit may further comprise reagents for use in the method/assay of the invention. For example, the kit may include phosphate-buffered saline containing 0.1% casein and/or 0.05% Tween-20 (PBST). The kit may further comprise adhesive film suitable for covering the wells during incubation to prevent or reduce evaporation. The kit may further include instructions for carrying out a meth/assay according to the invention.

### Prognosis/Diagnosis

The sample is obtained from the subject before the onset of EOPE, i.e. before symptoms arise or before health impacts arise for the mother and/or the fetus. Typically the sample is obtained in the first trimester of pregnancy in the subject, or within the first 12 or 16 weeks of pregnancy. The inventors have shown that even in the first trimester of pregnancy, increases in the quantity of CD10+CD63+, CD10+PLAP+ and CD63+PLAP+ sEVs in plasma or serum samples obtained from pregnant subjects are predictive of EOPE. Hence, a prognosis that a subject is at increased risk of going on to develop EOPE can be provided by comparing the quantity of CD10+CD63+, CD10+PLAP+ and/or CD63+PLAP+ sEVs in a sample obtained from the subject with reference quantities of CD10+CD63+, CD10+PLAP+, and/or CD63+PLAP+ sEVs, respectively. The reference quantities are typically the quantities detected in subjects who do not go on to develop EOPE, e.g. subjects who go on to have a normal pregnancy and/or subjects who go on to develop LOPE. Such reference quantities may be determined empirically. For example, in the Examples below, the average number of detected CD10+PLAP+ sEVs/μl serum sample was 1689 in subjects who went on to develop a normal pregnancy and 4521 in subjects who wenbenefitt on to develop EOPE. The quantity of CD10+CD63+ sEVs and the quantity of CD63+PLAP+ sEVs is likewise higher in subjects that go on to develop EOPE. The same was also seen in plasma samples. The skilled person is able to pre-determine a suitable threshold quantity above which the subject is determined to be at increased risk of going on to develop EOPE, based on previously obtained empirical data. Typically the quantity of sEVs having each pair of biomarkers is determined and compared to a separate reference quantity. Subjects who are determined, using the method/assay of the invention, to have higher quantities of CD10+CD63+, CD10+PLAP+ and/or CD63+PLAP+ sEVs than the respective reference quantities (for sEVs having each pair of biomarkers) may be determined to be at increased risk of EOPE. Typically, a higher quantity of any one of CD10+CD63+, CD10+PLAP+ and CD63+PLAP+ sEVs in the sample compared to a reference quantity may indicate that the subject has an increased risk of developing EOPE. In other cases, a higher total quantity of any two or all three of the CD10+CD63+, CD10+PLAP+ and CD63+PLAP+ sEVs compared to a single reference quantity may indicate that the subject has an increased risk of developing EOPE.

### Methods of Therapy and Medical Uses

The methods and assays described herein provide a novel window of opportunity to provide prophylactic treatment in early pregnancy which can be effective to prevent the later development of EOPE. The method/assay of the invention identifies the pregnant subjects that are most likely to benefit from the treatment. Hence, in some embodiments, the invention comprises carrying out the method/assay of the invention to prognose/diagnose/determine an increased risk of EOPE in a pregnant subject, and subsequently administering prophylactic treatment for EOPE to the subject determined to be at increased risk of EOPE.

The treatment may be the administration of aspirin, which is effective for preventing EOPE if administered within the first 16 weeks of pregnancy/gestation. Hence, the aspirin is administered, or aspirin in prescribed or aspirin administration is recommended for treatment of the patient, to commence within the first 16 weeks of pregnancy/gestation. Typically treatment may start within the first 14 weeks of pregnancy/gestation and/or from about weeks 11 to 14 of pregnancy/gestation. Hence, treatment may commence from about 11-14 weeks (77 to 98 days). The administration of aspirin is typically subsequently continued after 16 weeks of pregnancy/gestation. For example, treatment may continue up to about 36 weeks of pregnancy/gestation. A typical dose is about 150 mg of aspirin taken daily. Suitable regimes are described in Poon et al. (2019) Int. J. Gynaecol. Obstet. 145(Suppl 1):1-33.

In other cases the treatment may be (recommended/prescribed) administration of low molecular weight heparin (LMWH, for example as described in Roberge et al. (2016) Ultrasound Obstet, Gynecol. 47(5): 548-53), or the treatment may be (recommended/prescribed) administration of calcium supplement (for example as described in Thangaratinam et al. (2011) Best Pract. Res. Clin. Obstet Gynaecol. 25(4):419-33).

The invention further provides a method of treatment of a pregnant subject for preventing EOPE. The method comprises administering prophylactic treatment for EOPE to the subject, wherein the subject has been prognosed as being at increased risk of EOPE using a method or assay of the invention as described herein. The treatment may be administration of aspirin, as described above, typically commencing within the first 16 weeks of pregnancy/gestation.

The invention further provides aspirin for use in a method of preventing early onset preeclampsia (EOPE) in a pregnant subject, wherein the method comprises administering the aspirin to the subject before (or commencing before) 16 weeks of pregnancy/gestation, wherein the subject has been prognosed as being at increased risk of EOPE using a method or assay of the invention as described herein.

The invention further provides the use of aspirin in the manufacture of a medicament for preventing early onset preeclampsia (EOPE) in a pregnant subject, wherein the subject has been prognosed as being at increased risk of EOPE using a method or assay of the invention as described herein. The medicament/aspirin is typically for administration to the subject (commencing) before 16 weeks of pregnancy/gestation.

The subject may be a human or a non-human animal. Non-human animals include, but are not limited to mammals, rodents (including mice and rats), and other common laboratory, domestic and agricultural animals, including dogs, cats, horses, cows, sheep, goats and pigs.

The treatment may be administered by any suitable route and means. Aspirin is typically taken orally as a capsule or tablet.

Dosages and dosage regimes can be determined within the normal skill of the medical practitioner responsible for administration or prescription of the treatment. The dose may be determined according to various parameters, especially according to the age, weight and condition of the patient to be treated; the route of administration; and the required regimen.

Administration is typically in a “prophylactically effective amount” or a “therapeutically effective amount” (as the case may be, although prophylaxis may be considered therapy), this being sufficient to provide benefit to the subject and/or the fetus. Hence, the treatment may be sufficient to result in a clinical response or to show clinical benefit, for example to cure disease, prevent or delay onset or progression of the disease or condition or one or more symptoms, to ameliorate or alleviate one or more symptoms, to induce or prolong remission, or to delay relapse or recurrence. In some cases the treatment is sufficient to prevent preterm birth, to prevent SGA (small for gestational age) births/babies, or to prevent fetal or maternal death.

### Use of an Exocounter

In some embodiments, the invention provides the use of an ExoCounter device in a method of prognosing an increased risk of early onset preeclampsia (EOPE) in a subject. ExoCounter devices are described in WO 2014/168020 A1 and (15). The ExoCounter device may be used in any method or assay of the invention as described herein. The method may comprise incubating a plasma or serum sample obtained from the subject with anti-CD63, anti-CD10 and/or anti-PLAP sEV-capture antibodies. The capture antibodies may be coated on the surface of an optical disc (suitable for use with the ExoCounter device). The optical disc may have any suitable features described herein. The optical disc may comprise a plurality of wells. The base of each well may comprise a plurality of grooves, wherein the capture antibodies are coated (at least) on the base of the grooves. The incubation conditions are suitable for binding of sEVs in the sample to the capture antibodies in the grooves. The method may further comprise washing un-bound sample from the disk. The method further comprises incubating the captured sEVs in the grooves with detection nanobeads (wherein the detection nanobeads are suitable for use with the ExoCounter device). The nanobeads may have any suitable features described herein. The detection nanobeads may be coated with anti-CD63, anti-CD10 or anti-PLAP sEV-detection antibodies. The target antigen (CD63, CD10 or PLAP) of the capture antibodies in a well is different from the target antigen (CD63, CD10 or PLAP) of the detection antibodies coated on the nanobeads incubated in the same well. The incubation conditions are suitable for binding of detection antibody coated on single nanobeads to single sEVs in the grooves. The method may further comprise washing un-bound nanobeads from the disk. The method may further comprise counting the number of nanobeads bound to sEV in the grooves to quantify the detected absolute numbers of CD10+CD63+, CD10+PLAP+ and/or CD63+PLAP+ sEVs in the sample. This is typically done using a laser diode and a photodetector as described elsewhere herein. The method may further comprise comparing the detected absolute numbers of CD10+CD63+, CD10+PLAP+ and/or CD63+PLAP+sEVs of step with reference quantities of CD10+CD63+, CD10+PLAP+ and CD63+PLAP+ sEVs. The reference quantities are as described elsewhere herein. The method may further comprise determining an increased quantity of CD10+CD63+, CD10+PLAP+ and/or CD63+PLAP+ sEVs in the sample compared to the respective reference quantities of CD10+CD63+, CD10+PLAP+ or CD63+PLAP+ sEVs. The method further comprises prognosing an increased risk of early onset preeclampsia (EOPE) in the pregnant subject, wherein the increased risk is indicated by the determination in step (VII).

### EXAMPLES

### Example 1—psEVs Characterization

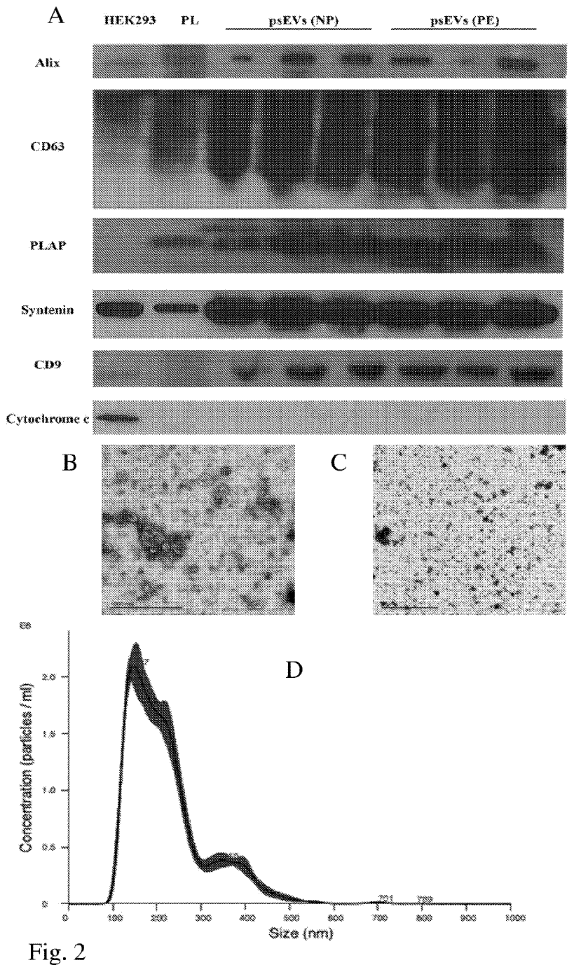

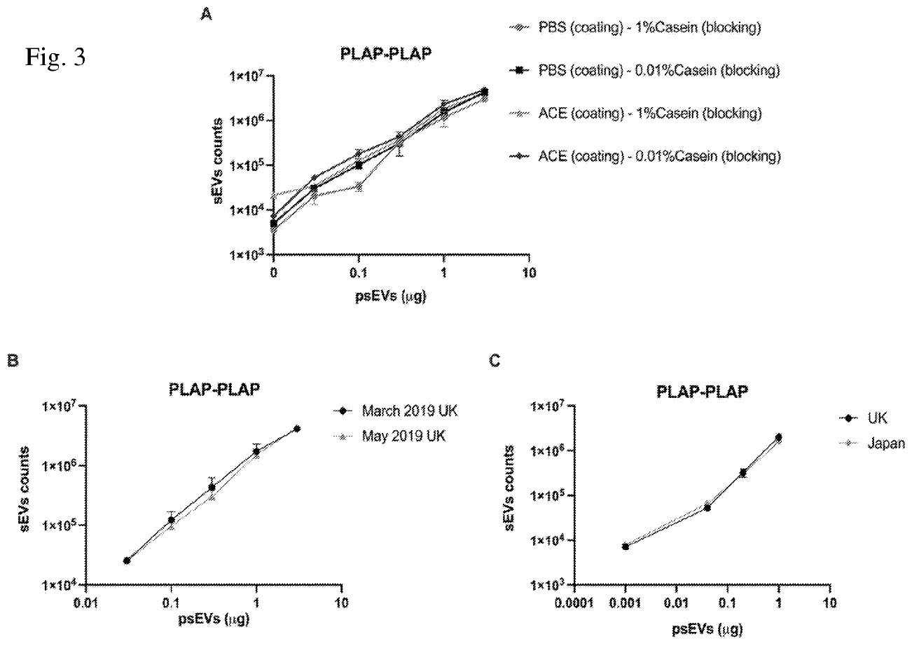

psEVs isolated from placental perfusion from NP and EOPE were characterized by TEM, western blot and NTA. psEVs expressed syntenin, CD63, Alix, CD9 (exosomal markers), PLAP (the syncytiotrophoblast origin marker), and were negative for cytochrome C (negative control) expression (FIG. 2A). Equal amounts of 20 μg protein as determined by BCA assays was loaded into each lane. Under TEM, psEVs exhibited a characteristic cup shape in negative staining (FIGS. 2B&C). NTA analysis revealed the mean of the size distribution of psEVs was (220.7±90) nm (FIG. 3D). PLAP was selected as a placenta-specific marker.

### Example 2—Antibody Coating and Blocking Buffer Optimization for ExoCounter Assay Using psEVs

The optimal experimental conditions for detecting psEVs obtained from placental perfusion using an ExoCounter were investigated, i.e., assay conditions which could yield the highest specific psEV counts, lowest background and highest assay linearity. Different coating antibody diluents and blocking buffers were tested. PLAP coating antibody and PLAP beads antibody were used to detect PLAP-PLAP psEVs since PLAP was proven to be abundantly expressed on psEVs (FIG. 2). ACE or fPBS were tested as coating antibody diluent and 1% Casein or 0.01% Casein in PBST were tested as blocking buffer. fPBS as coating antibody diluent and 0.01% Casein in PBST as blocking buffer were fund to yield a relative higher counts and lower background signals and the highest R²=0.9985 in linear regression analysis compared to other combinations (FIG. 3A). Therefore, fPBS was chosen as coating antibody diluent and 0.01% Casein was selected as blocking buffer for psEVs isolated from placenta perfusion.

Next, with the application of this optimized assay condition, the reproducibility of ExoCounter was tested. Experiments were conducted in the same Oxford laboratory at different dates using the same psEVs samples. Counts of PLAP-PLAP by different operators on different dates (FIG. 3B) were highly consistent. PLAP-PLAP psEVs counts appeared to reach the upper plateau phase from 1-3 μg but the lower plateau phase was not shown that the counts were still within the linearity range at 0.03 μg (FIG. 3B). Therefore it was decided to perform ExoCounter assay using protein concentration from 0.001-1 μg to check and determine the lowest quantifiable linearity range. The same sample was shipped on dry ice to Japan and analyzed under the same condition. Counts of PLAP-PLAP psEVs obtained in different organizations were again highly consistent (FIG. 3C). The data also suggested PLAP-PLAP psEVs counts appeared to reach the lower plateau phase from 0.04 μg to 0.001 μg (FIG. 3C).

### Example 3—the Most Abundant Tetraspanin of psEVs is CD63 Compared to CD9 and CD81, as Determined by MACSPlex Kit and Confirmed by ExoCounter

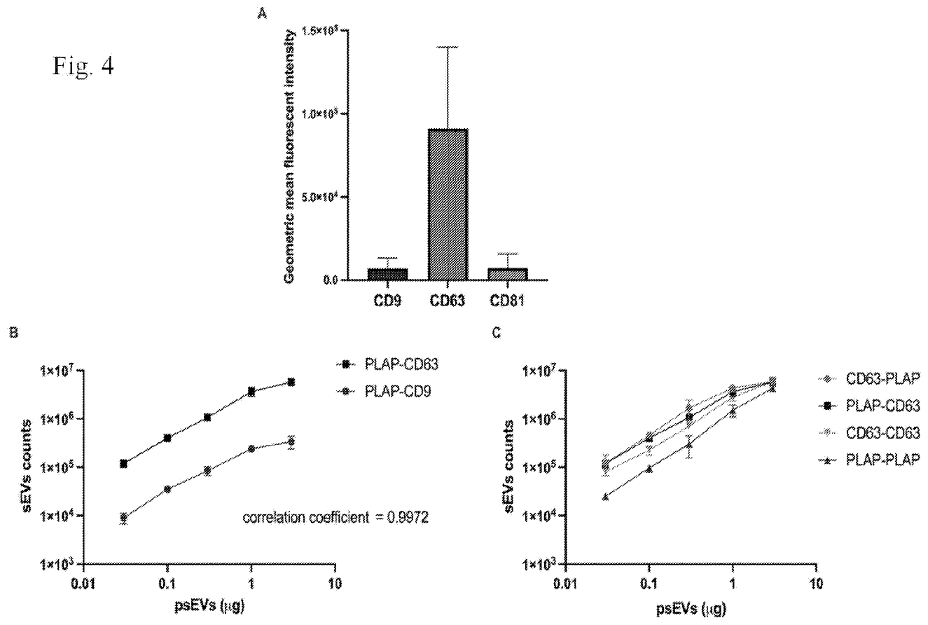

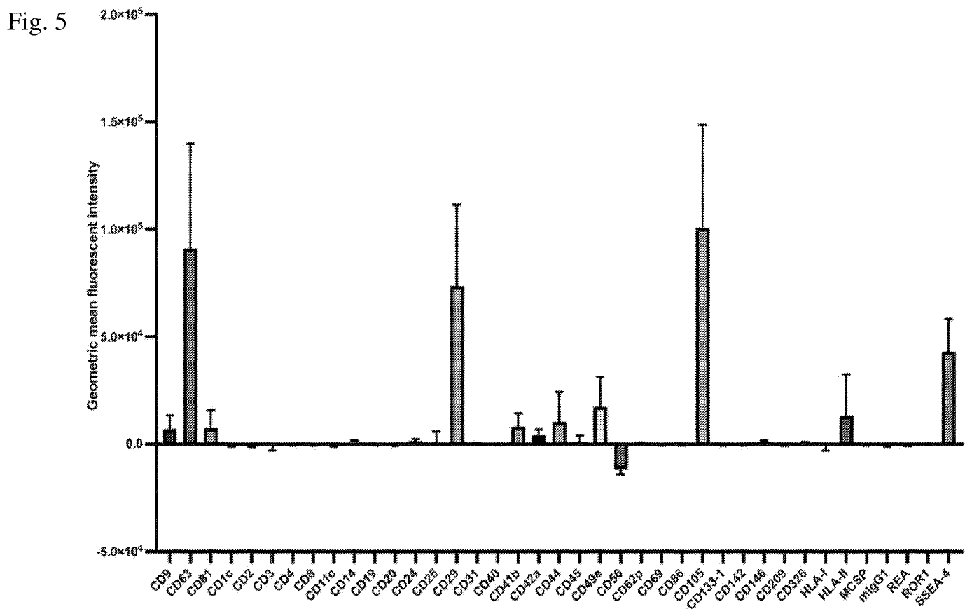

MACSplex kit were used for the identification of major PLAP-associated tetraspanin species on psEVs. Tetraspanins have been used as a surrogate marker for sEVs. Knowing which one of the tetraspanins is expressed in association with PLAP would allow for a more focused analysis of psEVs. To this end, MACSPlex was used on four psEVs samples isolated from placenta perfusion with a modification as specified in the method. The result showed net PLAP fluorescence in association with the 37 markers. Among the three tetraspanins CD9, CD63 and CD81, the highest PLAP fluorescence was found in association with CD63, indicating CD63 was the most common and abundant tetraspanin expressed by psEVs (FIG. 4A). For this reason, CD63 was used as a psEVs marker. The association of PLAP with the other 34 lineage markers of psEVs detected by MACSPlex is shown in FIG. 5.

Having found the differences between CD63 and CD9 in their association with PLAP using the MACSPlex kit, the association between tetraspanins CD63 or CD9 with placental marker PLAP by ExoCounter was tested next. Consistent with the MACSPlex results (FIG. 4A), counts of psEVs by PLAP-CD63 were higher than PLAP-CD9 (FIG. 4B). When using PLAP as capturing antibody, the counts of both PLAP-CD63 and PLAP-CD9 increased proportionally with the increasing psEVs protein concentration and the correlation coefficiency (Corr) between these two data sets was 0.9972 (FIG. 4B). Corr measures the normalized covariance of two data sets with a value of 1 indicating a perfect positive linear relationship. The Corr of PLAP-CD63 and PLAP-CD9 was close to 1 indicating ExoCounter was able to efficiently capture psEVs using PLAP as capturing antibody, and differences between PLAP-CD63 and PLAP-CD9 reflected quantitative differences in number of psEVs expressing CD63 and/or CD9. Taken together, both ExoCounter and MACSPlex assay consistently showed CD63 is the most abundant tetraspanin molecule co-expressed with PLAP. For this reason, CD63 was chosen to pair with PLAP for ExoCounter assay to represent psEVs in subsequent experiments. In addition, compared to MACSPlex, ExoCounter was able to generate an absolute count of psEVs thereby revealing quantitative differences for each protein concentration tested.

### Example 4—ExoCounter to Identify the Coating and Detecting Antibody Combination for psEVs Quantitation

Having established CD63 is the major tetraspannin molecule associated with PLAP, next it was tested if counts and backgrounds were affected by switching the same antibody pair from coating antibody to capturing antibody i.e. CD63-PLAP, PLAP-CD63, PLAP-PLAP and CD63-CD63 in ExoCounter assay (FIG. 4B). Counts from CD63 (coating)-PLAP (beads) combinations were highly consistent with PLAP (coating)-CD63 (beads) and CD63 (coating)-CD63 (beads). The counts for CD63-PLAP were slightly higher thus this combination was chosen for further experiments. Interestingly, the lowest counts were obtained using PLAP-PLAP combination (FIG. 4C). For all the combinations of antibodies tested, psEVs counts could be observed to reach the upper plateau phase when the amount of protein of psEVs tested was more than 1p g (FIGS. 4B & C). This confirmed the highest linear quantifiable limit of psEVs was 1p g for the combinations of antibodies tested. Taken together, the quantifiable linear range of the amount of protein of psEVs was determined to be 0.03 μg to 1 μg using ExoCounter. The linear regression formula within the quantifiable linear range for each antibody combination tested in FIG. 4C all have R² greater than 0.99 (Table 2).

TABLE 2
The linear regression formula and R² within the linear
quantifiable range from 0.03 μg to 1 μg for
each antibody combination tested on psEVs from FIG. 4C.

| Antibody Combination | Linear Regression Formula | R² |
|---|---|---|
| PLAP-CD9 | y = 234,076x + 8,894.3 | 0.9971 |
| PLAP-CD63 | y = 3,667,453.6x + 7,733.9 | 0.9997 |
| CD63-CD63 | y = 2,840,075.7x − 58,911.2 | 0.9980 |
| PLAP-PLAP | y = 1,567,370.5x − 74,457.8 | 0.9917 |
| CD63-PLAP | y = 4,294,819.8x + 115,132.7 | 0.9907 |

### Example 5—Selection of a PE-specific Biomarker

Mass spectrometry identified 1818 proteins in psEVs isolated from placenta perfusion. Of these, 73 proteins were differentially expressed in preeclampsia (PE) compared to normal pregnancy (NP) when tested for their expression levels by mass spectrometry. Of the tested markers, expression of COL17A1, FILAMIN B, Siglec6 and CLAUDIN4 were all significantly higher in preeclampsia (PE) compared to normal pregnancy (NP). These four markers were further shown to be upregulated in placentas from subjects who already had preeclampsia, in pm/lEVs (placental medium/large extracellular vesicles) and in placental small extracellular vesicles (psEVs) compared to NP using western blot (results not shown).

Antibodies against each of these four markers were tested as capture antibodies using an ExoCounter to detect psEVs isolated from placenta perfusion in subjects having a NP and those with PE. Two other capture antibodies were also tested, anti-DPP IV and anti-CD10, because the inventors have previously shown that expression of DPP IV on psEVs is upregulated in psEVs from gestational diabetes mellitus (GDM) patients compared to NP(1), that expression of DPP IV is found to be upregulated in PE placentas compared to NP placentas (2), and higher expression of CD10 on psEVs from PE compared to NP (3). Anti-PLAP was used as detection antibody coated on the detection nanobeads. Counts of PLAP co-expressed with COL17A1, FILAMIN B, Siglec6, CLAUDIN4, DPP IV or CD10 increased in a linear fashion corresponding to increasing concentration of psEVs protein concentration within the range of 0.03 μg-1 μg in both PE and NP. CD10 was selected as a PE-specific marker for further studies.

### REFERENCE

1. Kandzija N, Zhang W, Motta-Mejia C, Mhlomi V, McGowan-Downey J, James T, et al. Placental extracellular vesicles express active dipeptidyl peptidase IV; levels are increased in gestational diabetes mellitus. J Extracell Vesicles. 2019; 8(1):1617000.
2. Nishikawa M, Itakura A, Ito M, Takeuchi M, Sato Y, Kajiyama H, et al. Changes in placental dipeptidyl peptidase IV in preeclampsia with intrauterine growth restriction. Horm Metab Res. 2005; 37(7):408-13.
3. Gill M, Motta-Mejia C, Kandzija N, Cooke W, Zhang W, Cerdeira A S, et al. Placental Syncytiotrophoblast-Derived Extracellular Vesicles Carry Active NEP (Neprilysin) and Are Increased in Preeclampsia. Hypertension. 2019; 73(5):1112-9.

### Example 6—ExoCounter Detected Higher Placenta Specific CD63-PLAP sEVs Counts in Uterine Vein (UV) Compared to Pheripheral Vein (PV)

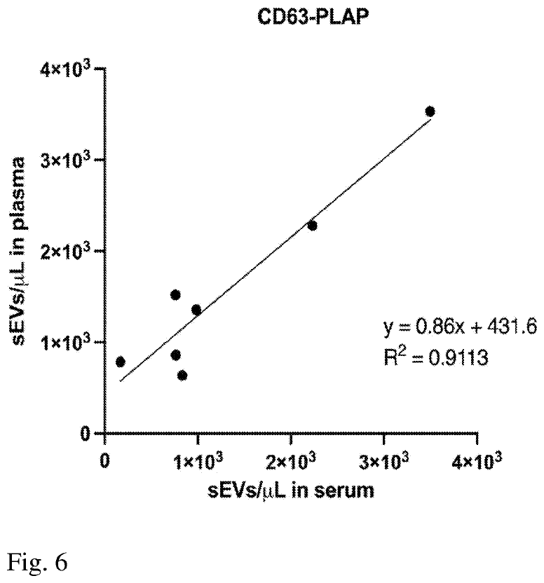

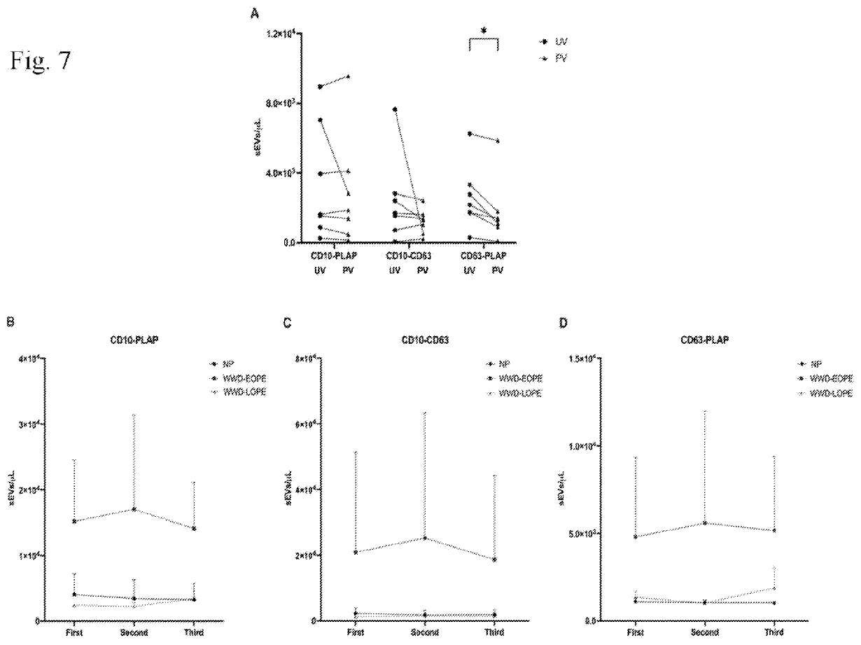

When optimized assay conditions were applied for perfusion generated psEVs to plasma samples from pregnant women, high background signals on ExoCounter were observed. The assay conditions were thoroughly optimised including the optimisation of blocking buffer and sample diluent (FIG. 6). After the optimisation for plasma samples, it was tested if sEVs counts by CD10-PLAP, CD10-CD63 and CD63-PLAP could be detected in plasma from normal pregnant women. The combination of CD10, PLAP and CD63 was chosen as described above: CD10 is a PE-specific disease marker on sEVs (13), PLAP is a placenta-specific marker and CD63 is a sEVs marker. The combination of either two markers from these three would give representatives of placenta-specific or disease-specific sEVs signals. The ExoCounter assay was validated by testing its ability to detect the known psEVs gradient between uterine and peripheral vein blood samples from the same donors. Indeed, for the 7 pairs of samples tested, the representative placenta-specific CD63-PLAP sEVs counts were all higher in the UV compared to PV (P<0.05, FIG. 7A). Either CD10-PLAP or CD10-CD63 sEVs counts didn't show a uniform gradient effect in UV and PV (FIG. 7A).

### Example 7—the Unique psEVs Profile of WWD-EOPE Compared to NP and WWD-LOPE Could be Observed as Early as the First Trimester

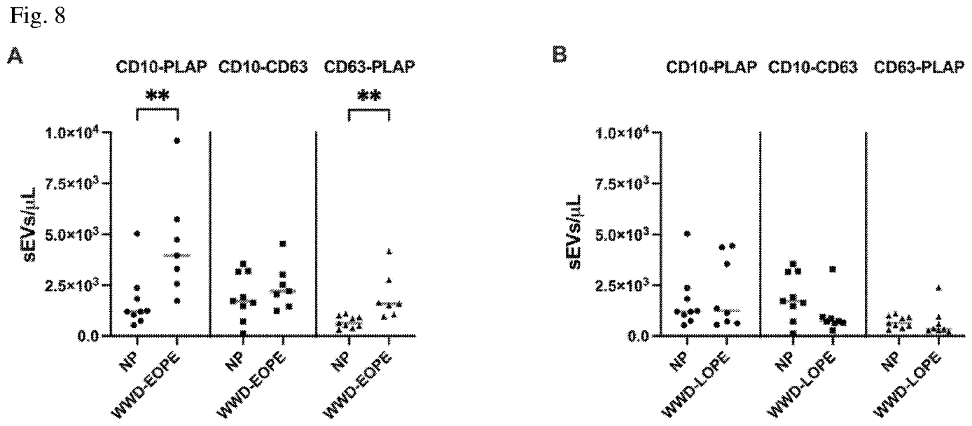

To see if there was a gestational-age-related and disease-specific change in psEVs, the counts of psEVs were quantified in longitudinal plasma samples from NP (n=3), EOPE (n=3) and LOPE (n=4), from first trimester (10-12 weeks), second trimester (20-21 weeks) and third trimester (29-31 weeks), using three antibody pairs: CD10-PLAP, CD10-CD63 and CD63-PLAP. When counts in plasma from the three trimesters of each individual patient were compared, none of the three combinations detected gestational age-related changes in counts with statistically significant differences (FIG. 8B, 8C, 10D). However, counts of psEVs as identified by all three coating and detection antibody pairs were elevated in WWD-EOPE compared to NP and WWD-LOPE as early as the first trimester. The high error bar observed might be due to the low sample size, but a clear separation between WWD-EOPE and NP or WWD-LOPE could still be observed.

The patient information for the plasma samples is shown in Table 3. The average systolic and diastolic blood pressure was significantly higher among the WWD-EOPE (163/100) and WWD-LOPE (155/96.8) groups compared to NP (118.8/65.3). Likewise, there was a significant difference in proteinuria and gestation age at delivery for WWD-EOPE and WWD-LOPE compared to NP. Finally, all the WWD-EOPE babies were born preterm and none of NP or WWD-LOPE baby were born preterm. There was no difference in maternal age, weight, body mass index (BMI) and smoking status.

TABLE 3
Patient's information for plasma samples.

| | Normal | WWD- | WWD- | P |
|---|---|---|---|---|
| Characteristics | Pregnancy | EOPE | LOPE | Value |
| Sample size | 3 | 3 | 4 | |
| Maternal | 26 ± 7.23 | 30.3 ± 7.23 | 26.2 ± 4.35 | 0.6533 |
| age years | | | | |
| (mean ± SD) | | | | |
| Maternal weight | 75.8 ± 24.2 | 69.1 ± 6.47 | 72.55 ± 1.97 | 0.8312 |
| kilograms | | | | |
| (mean ± SD) | | | | |
| Maternal height | 169.4 ± 5.31 | 169.1 ± 3.9 | 161.9 ± 8.8 | 0.3006 |
| centimetres | | | | |
| (mean ± SD) | | | | |
| Body mass | 26.7 ± 6.18 | 24.1 ± 1.77 | 27.8 ± 3.64 | 0.5420 |
| index kg/m−2 | | | | |
| (mean ± SD) | | | | |
| Times of gravidity | 1.7 ± 0.58 | 1.3 ± 0.58 | 2 ± 1.41 | 0.7061 |
| (mean ± SD) | | | | |
| Systolic blood | 113.3 ± 9.45 | 163.3 ± 15.28 | 155 ± 5.77 | 0.0011 |
| pressure mmHg | | | | |
| (mean ± SD) | | | | |
| Diastolic blood | 67 ± 6.24 | 100 ± 10 | 96.8 ± 3.77 | 0.0009 |
| pressure mmHg | | | | |
| (mean ± SD) | | | | |
| Proteinuria plus(es) | 0 | 2.7± 1.15 | 3.3 ± 0.5 | 0.0013 |
| (mean ± SD) | | | | |
| Gestational age at | 39.7 ± 0.58 | 33 ± 2.65 | 38.5 ± 1.29 | 0.0037 |
| delivery in weeks | | | | |
| (mean ± SD) | | | | |
| Birth weight grams | 3459.3 ± 352.1 | 1843.3 ± 491.74 | 3219.0 ± 673.49 | 0.0163 |
| (mean ± SD) | | | | |
| Preterm delivery (%) | 0 (0) | 3 (100) | 0 (0) | 0.0067 |
| Smoking History | 0/3 | 1/3 | 2/4 | 0.3564 |
| Intrauterine | 0 (0) | 3 (100) | 1 (25) | 0.0321 |
| growth restriction | | | | |
| (IUGR) (%) | | | | |
| Male gender (%) | 2 (66.7) | 0 (0) | 3 (75) | 0.1146 |

### Example 8—Significant Higher Counts of CD10-PLAP and CD63-PLAP psEVs were Detected by ExoCounter in WWD-EOPE Compared to WWD-LOPE and NP in the First-Trimester Serum Samples

The above Examples describe a unique psEVs profile of WWD-EOPE compared to NP and WWD-LOPE in the first trimester. These finding were further validated using increased sample sizes and biobank serum samples instead of plasma samples. The feasibility of using serum samples to validate was supported by the psEVs counts in plasma and serum identified by ExoCounter were highly consistent in the same donor (FIG. 6). From this collection of first-trimester serum samples, NP (n=9), EOPE (n=7) and LOPE (n=8) were selected for matched BMI, parity and disease severity. Only severe PE patients without HELLP syndrome were selected. Severe PE is defined according to the International Classification of Diseases (ICD) as blood pressure over 160/110 mmHg and required hospital admission. Other patient's demographics and clinical characteristics are shown in Table 4.

Significantly higher psEVs counts were found in the first-trimester serum samples from WWD-EOPE compared to NP detected by CD63-PLAP (P<0.01) and CD10-PLAP (P<0.01, FIG. 8A). There is no significant difference of psEVs counts detected by CD10-CD63 from WWD-EOPE compared to NP. None of the three pairs, CD10-PLAP, CD10-CD63 and CD63-PLAP, showed significant differences between NP and WWD-LOPE in the first-trimester serum samples (FIG. 8B).

TABLE 4
Patient's information for serum samples.

| | Normal | | | P |
|---|---|---|---|---|
| Characteristics | Pregnancy | EOPE | LOPE | Value |
| Sample size | 9 | 7 | 8 | |
| Maternal | 29 ± 8.78 | 28.1 ± 3 | 31.3 ± 6 | 0.6555 |
| age years | | | | |
| (mean ± SD) | | | | |
| Body mass | 25.0 ± 3.8 | 25.3 ± 3.3 | 24.0 ± 2.5 | 0.7126 |
| index kg/m−2 | | | | |
| (mean ± SD) | | | | |
| Birth weight | 3340 ± 394 | 1282.3 ± 390.2 | 3293.1 ± 669.5 | <0.001 |
| grams | | | | |
| (mean ± SD) | | | | |
| Smoking History | 3/9 | 1/7 | 3/8 | 0.5784 |
| Male gender (%) | 3 (33) | 1 (14) | 5 (62.5) | <0.001 |

### Example 9—ExoCounter Assay Optimization for Plasma Samples

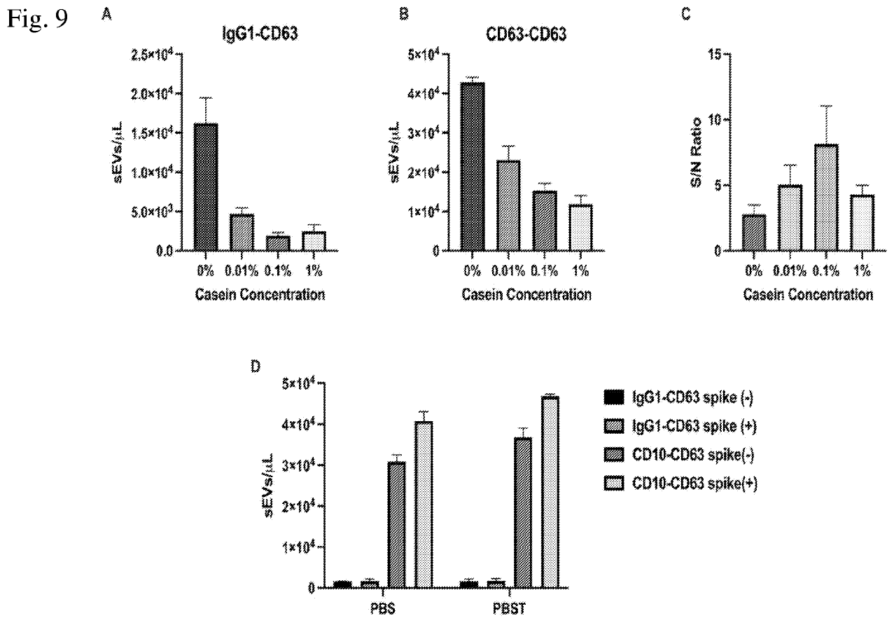

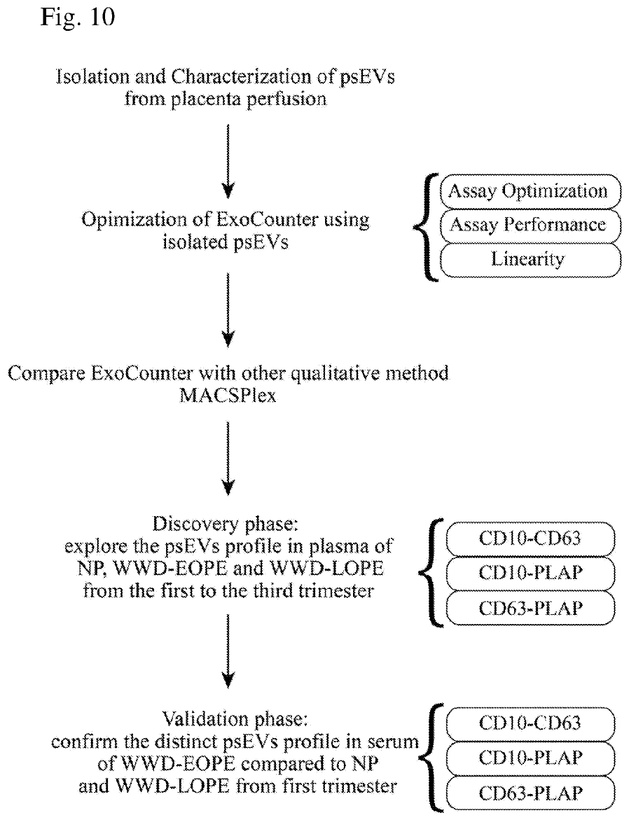

When optimized assay conditions were applied for perfusion generated psEVs to plasma samples from pregnant women, very high background signals on ExoCounter were observed. Plasma samples were diluted in sample diluent at 1 to 4 ratio and 12.5 mL of plasma was used per well. The initial sample diluent used was PBS. The inventors reasoned that, unlike placental perfusion derived psEVs, plasma samples contain high levels of immunoglobulin (IgG). Non-specific binding between plasma IgG and beads might be a cause of high background observed. In the past, Tween-20 and casein were shown to be effective to reduce non-specific binding in enzyme-linked immunosorbent assays using plasma (Kenna et al. (1985) J. Immunol. Methods 85(2): 409-19). The inventors therefore reassessed casein concentration in the beads antibody diluent for reduction of non-specific binding. To identify the optimal concentration of casein for background reduction, different concentrations of casein (0%, 0.01%, 0.1%, 1%) were added to the beads antibody diluent. Counts from IgG1-CD63, an indication of non-specific binding in plasma samples, were compared. The counts of IgG1-CD63, decreased with increasing concentration of casein in beads diluent with 0.1% casein yielding the lowest background counts (FIG. 9A). Next, the effect of casein in the beads antibody diluent on CD63-CD63 specific signals from the same plasma samples was checked. CD63-CD63 was chosen instead of CD63-PLAP since CD63-CD63 was more likely to capture not just psEVs but sEVs from other cellular sources in plasma samples. Second, CD63 antibody conjugated beads were found to generate higher background signals compared to PLAP antibody-conjugated beads (data not shown). After casein was added to the beads diluent, CD63-CD63 sEVs counts were found to be also decreased with increased concentration of casein (FIG. 9B). The highest signal to noise ratios (dividing the counts from CD63-CD63 by IgG1-CD63) were evaluated, and the highest signal to noise ratio was seen when 0.1% casein was used as beads antibody diluent (FIG. 9C). Thereafter, 0.1% casein was used in beads diluent for plasma samples.

However, with application of 0.1% casein in beads diluent to the three antibody combinations CD10-PLAP, CD10-CD63 and CD63-PLAP to analyze placental sEVs in plasma samples from pregnant women, high background signals from CD10-CD63 (data not shown) were again observed. Next it was tested if background in plasma could be further reduced by adding 0.05% of Tween 20 to PBS (PBST) and using it as sample diluent instead of just PBS. Furthermore, it was checked if CD10-CD63 sEVs were only specific to pregnant women (i.e., only detectable in pregnant plasma samples and not detectable in non-pregnant plasma samples). And if CD10-CD63 sEVs were not specific to plasma samples from pregnant women, if it was possible to detect the contribution of placenta specific CD10-CD63 sEVs signals from plasma samples. For these purposes, a 1 in 4 dilution of plasma samples from non-pregnant women was made in either in PBS or in PBST, and psEVs at 4 mg/mL were used to spike in non-pregnant plasma samples. If placenta-specific CD10-CD63 signals could be detected, higher counts would have been seen in psEV spiked plasma samples.

Low levels of counts were obtained from IgG1-CD63 regardless of whether PBS or PBST was used as sample diluent, and with or without spiked in psEVs (FIG. 9D). In contrast, CD10-CD63 counts in non-spiked samples were 20 times higher than IgG1-CD63 in plasma samples, indicating these CD10-CD63 signals from plasma samples were not pregnancy-specific. After psEVs were spiked in the plasma samples, higher counts (1×10⁵ difference) have been seen in psEV spiked plasma samples compared to samples without spike-in when PBST was used as sample diluent (FIG. 9D). These results indicated placenta specific CD10-CD63 psEVs from the spiked in was detected above the existing CD10-CD63 sEVs signals from plasma samples by ExoCounter. Overall, PBST yielded higher specific signals compared to PBS. Thus, PBST was chosen as plasma diluent in the subsequent experiments.

### Example 10—Counts of psEVs Detected by ExoCounter were Highly Consistent in Serum and Plasma Samples from the Same Donor

Before moving from plasma samples to serum samples to validate the results acquired from plasma samples (FIGS. 7B & 7C & 7D), it was checked if the psEVs counts detected by ExoCounter would be influenced by sample type, i.e., serum or plasma. For this purpose, 7 paired third-trimester serum and plasma samples from the same NP donor were used. The psEVs counts detected by CD63-PLAP in serum of all 7 pairs were similar to the counts in plasma (FIG. 6). This indicates the psEVs counts detected by ExoCounter would not be influenced by the sample type.

### Methods

### Human Subjects and Ethic Approval

We obtained ethical approval from the Central Oxfordshire Research Ethics Committee C (REFS 07/H0607/74 & 07/H0606/148 & 08/H0606/139). For placental perfusion, we prospectively selected pregnant women undergoing elective caesarean sections before labour onset, from the maternity ward at the Women's Centre, John Radcliffe Hospital, Oxford. The placentas were subsequently collected from normotensive (NP) and preeclamptic (PE) women and perfused within 10 to 20 minutes of delivery. We obtained written informed consent before placenta collection from all participants. Platelet poor plasma taken at the first (10-12 weeks), second (20-21 weeks) and third trimesters (29-31 weeks) were retrieved from the biobank (Ethics as above). We defined normal pregnancy (NP) as a singleton pregnancy with no history of preeclampsia, hypertensive disorders, or other complications in pregnancy. Patients with preeclampsia were defined as displaying the co-occurrence of de novo hypertension (blood pressure >140/90 mmHg) and proteinuria (>300 mg/day) after week 20 of gestation according to the criteria of the International Society for the Study of Hypertension in Pregnancy. The EOPE was defined as the onset before 34 weeks and LOPE was defined as the onset on or after 34 weeks of gestation.

Isolation of psEVs by Placental Dual-Lobe Perfusion and Differential Ultracentrifugation

Methods for isolation and enrichment of psEVs through ex-vivo dual lobe perfusion have been described in detail in a previous publication (16). Briefly, we identified a suitable cotyledon devoid of calcifications, ischemia, or rupture. We identified and cannulated a suitable placental artery and vein and perfused the placenta for three hours at a 4-5 mL/min flow rate to obtain placenta maternal perfusate. The placenta maternal perfusate was centrifuged twice at 1,500 g for 10 minutes at 4° C. (Beckman Coulter Avanti J-20XP centrifuge using a Beckman Coulter JS-5.3 swing-out rotor) to remove cells and cell debris. The Supernatant was carefully pooled and spun at 10,000 g (10K) in a swing bucket centrifuge (Beckman L80 ultracentrifuge and Sorvall TST28.39 swing-out rotor) at 4° C. for 30 minutes. The Post-10K supernatant was filtered through a 0.22 μm Millipore Stericup Filtration Device (Merck, SCVPU11RE), before being spun again at 150,000 g for 2 hours (Beckman L80 ultracentrifuge with a Sorvall TST28.39 swing-out rotor). The post 150K pellet was washed and resuspended in 0.1 μm filtered calcium- and magnesium-free phosphate buffer saline PBS (fPBS) to a protein concentration of approximately 2-5 μg/μL (measured using a Pierce BCA protein assay), and immediately stored at −80° C. as 25-50 μL aliquots. All the medium used in placenta perfusion and sample processing were filtered using a 0.1 μm filter device before use. A fresh stock of psEVs was thawed and then used for subsequent analysis.

### Plasma Sample Preparation

Sodium citrate platelet free plasma samples from the first (10-12 weeks), second (20-21 weeks) and third trimester (29-31 weeks), were retrieved from the biobank. All samples had been collected following a standardised plasma sample protocol. In brief, blood was collected into sodium citrate vacutainers (BD Biosciences) and transported to the lab within 30 minutes for sample processing. To obtain plasma, vacutainers were centrifuged at 1,500 g for 15 minutes at room temperature in a swing bucket centrifuge (Beckman Coulter Avanti J-20XP centrifuge using a Beckman Coulter JS-5.3 swing-out rotor). Plasma from 0.5 cm above the buffy coat layer was collected, aliquoted to Eppendorf tubes and further centrifuged at 13,000 g for 2 mins in a fixed angle desktop microfuge. After this last centrifugation, 450 μL of platelet free plasma (PFP) from each Eppendorf tube, was transferred to a new Eppendorf tube leaving behind 50 μL of plasma and pellet to minimize platelet contamination. PFP was stored immediately at −80° C. On the day of the experiments, the samples were defrosted and spun at 13,000 g for 2 mins before investigation on ExoCounter. In total, 3 NPs, 3 EOPEs and 4 LOPEs were analyzed on the ExoCounter.

For the uterine vein (UV) blood and peripheral vein (PV) experiments, the UV blood was collected by the surgeon during the caesarean section before the delivery of the baby, using a 20 ml syringe with a 21G needle. The blood was then transferred to a sodium citrate tube. The PV blood was collected using a sodium citrate vacutainer just before the caesarean section. Both UV and PV blood were processed according to the standardised plasma sample protocol as stated above. PFP from these samples was then frozen at −80° C.

### Serum Sample Collection

The first-trimester serum samples were part of the Thames Valley Collection. This collection includes a collection of first-trimester serum samples used for the routine triple test from Oxford University Hospitals NHS Foundation Trust, Buckinghamshire Healthcare NHS Trust, and Milton Keynes University Hospital from May 2015 to August 2018. Each sample was retrospectively coded according to the International Classification of Disease (ICD). For WWD-EOPE and WWD-LOPE, only patients with the code of 0141 indicating severe PE were selected. Severe PE is defined according to ICD as blood pressure over 160/110 mmHg, required hospital admission and without hemolysis elevated liver enzymes and low platelets (HELLP) syndrome. After the blood samples were collected, the blood samples were centrifuged at 3,000 g for 5 minutes and the serum was aliquoted into 0.5 ml aliquots and stored at −80° C.

### Western Blot

Following the guidelines by the International Society for Extracellular Vesicles (ISEV), all the psEVs obtained from placenta perfusion were characterised for the presence and absence of a panel of the following markers; PLAP (for syncytiotrophoblast origin, in-house antibody), CD63 (Santa Cruz Biotechnology, Sc-59286), CD9 (Abcam, ab92726), syntenin (Abcam, ab133267) and ALIX (Santa Cruz Biotechnology, Sc-53538, to confirm the presence of extracellular vesicles, all antibodies at 1 in 1000 dilution), and Cytochrome C (as negative EV marker, 1 in 1000 dilution, Santa Cruz Biotechnology, Sc-13156). HEK293 cell lysate and placental tissue lysates each worked as positive and negative controls for EV markers and syncytiotrophoblast marker. Lysates were made using RIPA buffer (ThermoFisher, 89901) and the protein concentrations were determined using Bicinchoninic acid (BCA) assay (ThermoFisher, 23227). In brief, 20 μg of protein of placental sEVs were mixed with 4× Laemmli buffer with (for PLAP, Cytochrome C, syntenin) or without (for CD63, CD9, Alix) 5% β-Mercaptoethanol and diluted to 20 μL with distilled water. The sample mix was heated at 70° C. for 10 mins before loading onto a 4%-12% SDS-PAGE 10-well gel (ThermoFisher, NP0321BOX) for electrophoresis. Proteins were transferred onto PVDF membranes (Bio-Rad, 1620177) using a semi-dry transfer system. The membranes were then blocked for 1 h in 5% Blottto (2BScientific, AD-80402-500) in Tris-Buffered Saline with 0.1% Tween-20 (TBS-T, ThermoFisher) before incubation with primary antibody overnight at 4° C. After further incubation with the appropriate secondary antibody for an hour, chemiluminescence (ECL) (Thermo Fisher, 32106) was used for signal enhancement and developed in a dark room (Hyperfilm ECL, Merck, GE28-9068-35).

### Transmission Electron Microscopy (TEM)

psEVs isolated from perfusion were diluted with fPBS to achieve a concentration between 0.1-0.3 μg/μL. psEVs were fixed with 2% paraformaldehyde to preserve the morphology and internal structure if samples were left overnight before negative staining. 10 μL of the psEVs solution was applied to freshly glowing discharged carbon formvar 300 mesh copper grids for 2 mins, blotted with filter paper, and stained with 2% uranyl acetate for 10s, then blotted and air-dried. psEVs on the grid were negatively stained to enhance the contrast between psEVs and the background. The grids were imaged using an FEI Tecnai 12 TEM at 120 kV with a Gatan OneView CMOS camera.

### Nanoparticle Tracking Analysis (NTA)

We further characterized the psEVs isolated from placenta perfusion by nanoparticle tracking analysis [(NTA) NanoSight NS500 instrument equipped with a 405 nm laser (Malvern UK), sCMOS camera and Nanoparticle Tracking Analysis (NTA) software version 2.3, Build 0033 (Malvern UK)]. Before sample analysis, instrument performance was checked with silica 100 nm microspheres (Polysciences, Inc.). The samples were diluted in fPBS to a range of 1/10,000 to 1/100,000 as determined by the starting concentration. The samples were automatically injected into the sample chamber with a 1 ml syringe with the following script used for EV measurements: PRIME, DELAY 5, CAPTURE 60, REPEAT 4. Images of the analyzed samples were captured on camera at a level of 12 (Camera shutter speed; 15 ms and Camera gain; 350) and NTA post-acquisition settings were optimized and kept constant between samples. Each video recording was analyzed to infer placental sEVs size and concentration profile.

### Detection of PLAP-Associated Antigens by MACSPlex Exosome Kit

The MACSPlex exosome kit (Miltenyi Biotech, 130-108-813) was used with modification to probe psEVs antigens and their association with PLAP. Briefly, 10 μg of psEVs isolated from placenta perfusion were incubated with MACSPlex Exosome Capture beads following the manufacturer's short protocol for 1.5 mL reagent tubes. Samples were washed to remove the non-specific binding and unbound EVs at the end of incubation. Instead of using MACSPlex Exosome Detection Reagents (APC-conjugated CD9, CD63 and CD81 antibodies), APC-conjugated PLAP antibody (an in-house mouse IgG1 antibody) at 0.5 μg/mL or APC-conjugated IgG1 isotype control (Biolegend) at 1 in 1,000 was added. The remaining incubation and sample washing steps were conducted following the standard manufacturer's protocol. Samples were analyzed using Becton Dickinson LSR-II flow cytometer. For data analysis, the net geometric means of PLAP fluorescence from each capture bead were calculated by subtracting correspondent APC-conjugated IgG1 isotype stained control tubes from the geometric means fluorescence of the APC-conjugated PLAP antibody stained tubes. The fluorescent intensity was an indication of PLAP association with antigens captured by capture beads. The higher the fluorescence, the more abundant PLAP antigens.

### ExoCounter

The ExoCounter (JVCKENWOOD corp.) can capture and quantify the sEVs by sandwich immunoassay using disc and secondary antibodies conjugated to nanobeads (FIG. 1). Unique to ExoCounter is that 1) disc is engraved with circular U-shaped trenches 260 nm in width at the top and 160 nm at the bottom. The surface of the disc allows for coating of capturing antibody which permits sEVs <160 nm to be captured at the bottom of the trench; 2) Captured sEVs are further recognized by the secondary antibody conjugated to magnetic nanobeads (FG beads) (17). The size of the nanobeads is 200 nm which permits the beads to enter the trench to bind with sEVs while preventing a second bead from entering. This allows one bead to bind to one sEV. The numbers of beads were counted by an optical pickup equipped with a laser diode, which can illuminate the disc, and a photodetector which can detect the light reflected from the disc. The light from the beads attached to the exosomes was transformed into pulses and counted by a pulse counter circuit. The number of pulses generated by diffraction is therefore equal to the number of captured and labeled sEVs; 3) the same or different antibodies can be used in combination giving the flexibility for interrogation of up to two antigens per well. In order to easily describe the experimental antibody layout used for the ExoCounter experiments in the current study, the form of (antibody-1)-(antibody-2) is used to describe the coating antibody (antibody-1, in the groove) and bead-bound antibody (antibody 2). For example, CD63-PLAP denotes CD63 as coating antibody and PLAP as beads antibody.

This assay has five major steps. First, 90 μL of coating antibody (5 μg/mL) was applied on a well of the disc to coat for 1 h at 37° C. Next, 200 μL of blocking buffer was applied to block non-specific signals for 1 h at room temperature. Then, 50 μL of samples was applied to the disc and incubated for 2 hs at 37° C. on an orbital shaker. For psEVs from perfused placentas, psEVs were diluted according to the desired concentrations using fPBS up to 50 μL. For plasma or serum samples, 12.5 μL of plasma or serum samples were diluted in 37.5 μL fPBS or PBST to make up to the total 50 μL. Next, 50 μL of bead-bound antibody solution (20 μg/mL) was applied to the disc for 90 mins at 37° C. on an orbital shaker. Finally, after the washing and drying steps, the signals were read by the optical system using a Blu-ray laser device and each bead was counted as pulses by the pulse sensing circuit. Between each step, an automatic washer was used to remove the excess reagents and reduce non-specific binding. The washing buffer used was 0.05% Tween-20 in PBS (PBST). The washing program was set to wash the disc with 300 μL of PBST 3 times, and an additional wash with 3,000 μL deionized water to minimize salt crystal formation before disk drying.

For initial assay development, different coating dilution buffer (PBS or Acetate (ACE, 3863)) and disc blocking buffers (1% casein (Thermo Scientific, 37528) or 0.01% casein in PBST) were tested. The coating antibodies used in the experiments were listed in Table 1. Antibody bead conjugation was performed as previously described (15). For each bead antibody, 50 μL of bead antibody solution (20 μg/ml) contains approximately 1.6×10⁸ FG nanobeads per well. psEVs isolated from placental perfusion were used to validate the performance of ExoCounter. In brief, psEVs at 3 μg/well, 1 μg/well, 0.3 μg/well, 0.1 μg/well and 0.03 μg/well were diluted in fPBS and the signals obtained were compared. Each protein concentration was performed in triplicate which resulted in 15 wells being used and the last well (each disc has 16 wells in total) was used as blank well which was treated under identical conditions except no sample was added.

### Statistical Analysis

Statistical analysis was performed using GraphPad Prism 9. Continuous variables were compared using the Mann-Whitney test. Categorical variables were compared using the chi-square test. The difference between the uterine vein and peripheral blood was compared using the Wilcoxon test (paired non-parametric test).

### Experimental Design and Flow

The experimental design for the experimental study described herein is shown in FIG. 9. First, psEVs from placenta perfusion were isolated and characterized. Then, the isolated psEVs were used to optimize ExoCounter performance by testing different blocking buffers and antibody diluents. After the optimization, the assay linearity and repeatability of ExoCounter was measured and compared with another qualitative method, MACSPlex. Next, the longitudinal first-, second- and third-trimester plasma samples from NP (n=3), WWD-EOPE (n=3) and WWD-LOPE (n=4) were applied on ExoCounter using CD10-PLAP, CD10-CD63 and CD63-PLAP three pairs antibodies to determine if there were disease-specific or gestation-specific psEVs profile. The findings were validated by using a larger sample size of the first-trimester serum samples from NP (n=9), WWD-EOPE (n=7) and WWD-LOPE (n=8).

### Discussion

The inventors have developed and optimized assays to quantify and phenotype placental small EVs (psEVs) isolated from placental perfusion. The assay has been exemplified using the ExoCounter instrument. Adaptations have been made for interrogation of psEVs in plasma and serum samples. The ExoCounter assays 1) showed a wide linear range with high intra-organisation and inter-organisation reproducibility, 2) identified CD63 as the major tetraspannin associated with PLAP, 3) detected the previously identified gradient in psEVs between the UV and PB; 4) identified significant differences between WWD-EOPE and NP in the first-trimester serum samples. To knowledge, this is the first study to show psEVs counts e.g. by CD10-PLAP and CD63-PLAP differed significantly between NP and WWD-EOPE but not between NP and WWD-LOPE. And the differences can be detected as early as in the first trimester ((<12 weeks) plasma or serum samples. Previous studies found higher PLAP-positive placental EVs detected by ELISA in EOPE plasma samples compared to LOPE and NP in the third trimester's samples (9, 18). However, the inventors are the first to surprisingly detect differences in the first trimester.

Previous studies have reported placental EVs in NP plasma increased with the advancing gestational age (2, 19). These results were not found in the present study. The following reasons may explain the difference. First, other studies have isolated placental particles prior to quantification. The ultracentrifugation (UC) method used in previous studies can co-sediment unwanted soluble proteins and other non-EV materials (2, 3, 10, 19). It has been suggested that only a minority of EVs could be isolated from plasma by UC (20). Taken together, it may not be appropriate to compare data generated from processed and un-processed samples. Secondly, the difference may be due to the size of the EVs quantified. In the present study, the physical structure of the discs selectively quantified placental EVs no larger than 160 nm. However, the size of placental EVs from the previous studies varies from 50 nm to 200 nm (2, 19) as detected by NTA. Thirdly, the antibodies for placental EV quantification differ between studies. For other studies, the placental EVs were quantified using anti-PLAP as the first antibody for ELISA (2, 3, 10, 19). In the present study, two pairs of antibodies, namely anti-CD63 and anti-PLAP were used to define not only their placental origin but also the expression of tetraspanins.

The inventors found the counts of CD10-PLAP psEVs was significantly elevated in WWD-EOPE compared to NP serum from the first trimester (FIG. 8A). CD10, also known as neprilysin, is a zinc metalloendopeptidase which is a pleiotropic inhibitor of several bioactive vasopeptides. Pharmacological inhibition of CD10 has been proven to be an effective treatment in hypertension, which is a hallmark of preeclampsia (21). The inventors have previously shown higher CD10 activity in sEVs released by preeclamptic placentas (13). The current study suggests CD10 may be involved in the development of EOPE many weeks before the onset of clinical features of disease. CD10 has been shown to have anti-angiogenic ability. The inventors proposed that elevated CD10-PLAP psEVs in EOPE plasma and serum, together with other well-known anti-angiogenic proteins including soluble fms-like tyrosine kinase (sFlt-1) and soluble endoglin (sEng), could play a role in maternal endothelial dysfunction seen in EOPE (22, 23).

Using the uterine vein and paired peripheral vein samples, the inventors have shown that CD63-PLAP psEVs were higher in uterine vein compared to peripheral blood (FIG. 12A). This is consistent with other studies that PLAP-positive EVs were higher in UV compared to PB. However, the counts of CD10-PLAP and CD10-CD63 psEVs didn't exhibit this gradient difference (FIG. 12A). This is possibly due to CD10-positive sEVs are not placenta specific. Apart from placenta, CD10 has a variety of origins such as heart, kidney, lung, brain and adipose tissue. The possible confounding of CD10-positive sEVs from other tissues releasing into the blood might prevent the gradient difference from the observation between PV and UV. Indeed, CD10-CD63 sEVs were also identified in the plasma of non-pregnant women by ExoCounter (FIG. 9D), supporting the presence of a high level of CD10-CD63 sEVs in plasma samples from other tissues.

The inventors recognized that ExoCounter possesses several unique features which mean it outperforms existing approaches to circulating placental EV quantification. First, it requires minimal sample processing. Other quantification methods which allow the interrogation of EVs markers, such as fluorescence NTA and flow cytometry, are highly dependent on complicated sample processing. For example, the BD Influx flow cytometry can interrogate sEVs subsets with fluorescent labeling, however, density gradient ultracentrifugation is recommended to separate the unbound dye during sample processing. Fluorescence NTA showed high background signals with unprocessed samples, suggesting many non-EV particles were detected (24). Complicated sample processing not only increases time required from processing samples to acquiring results, it is also likely to introduce procedure-operator-dependent biases. Secondly, the ExoCounter combines size exclusion with immunopreciptation, removing key confounders of each technique alone. For example, size exclusion approaches co-isolate chylomicrons and lipoproteins, which far outnumber psEVs in plasma. Conversely immunoprecipitation is hampered by co-isolation of protein targets which are not expressed on EVs, such as soluble proteins and cell fragments. Thirdly the Exocounter can interrogate two markers simultaneously, allowing the inventors to combine markers of vesicles, tissue origin and disease, dramatically improving the specificity of the assays. Fourthly the semi-automated setup of the ExoCounter reduces operator errors resulting in highly reproducible results (FIGS. 3B & 2C). Finally, the Exocounter works with very small sample volumes, making it highly attractive for use in clinical practice and opening the possibility of unskilled sample acquisition (e.g. capillary blood sampling), which widens its applicability whilst reducing the cost of the diagnostic sampling. The ExoCounter is a strong candidate for application in larger clinical studies.

In conclusion, the inventors have surprisingly demonstrated that CD10-PLAP and CD63-PLAP psEVs are effective biomarkers to diagnose WWD-EOPE from the first trimester of pregnancy. The inventors have further surprisingly demonstrated that the ExoCounter platform permits highly reproducible quantitative (counts) and qualitative (two antigens) sEVs analysis in minimally processed biological fluids. Distinct psEVs profiles were detected by ExoCounter in early gestation (<12 weeks) in EOPE compared to NP and LOPE. Hence, the invention described herein can be used for EOPE early prognosis and diagnosis, patients risk stratification and early intervention before the onset of the disease.

### LIST OF ABBREVIATIONS

ACE Acetate
ALIX ALG-2 interacting protein X
ANOVA Analysis of variance
BCA Bicinchonic assay
ECL Chemiluminescence
EOPE Early onset preeclampsia
ESCRT Endosomal sorting complexes required for transport
EVs Extracellular vesicles
fPBS Filtered phosphate buffered saline
HELLP hemolysis elevated liver enzymes and low platelets
ISEV International Society for Extracellular Vesicles
LOPE Late onset preeclampsia
mg Milligrams
mL Millilitres
MVE Multivesicular endosomes
NEP Neprilysin
NP Normal pregnancy
NTA Nanoparticle tracking analysis
PV Peripheral vein
PBS phosphate buffered saline
PBST Phosphate buffered saline with Tween-20
PE Preeclampsia
PLAP Placenta alkaline phosphatase
PL Placenta lysate
PFP Platelet free plasma
psEVs Placental small extracellular vesicles
PVDF Polyvinylidene difluoride
RBC Red blood cell
sEVs Small extracellular vehicles
sEng Soluble endoglin
sFlt-1 Soluble fms-like tyrosine kinase
STB Syncytiotrophoblast
TBST Tris-buffered saline with Tween-20
TEM Transmission electron microscopy
TSG101 Tumour susceptibility gene 101
UV Uterine vein
WB Western blot
μg Micrograms
μl Microlitres

### REFERENCES

1. Gurung S, Perocheau D, Touramanidou L, Baruteau J. The exosome journey: from biogenesis to uptake and intracellular signalling. Cell Commun Signal. 2021; 19(1):47.
2. Sarker S, Scholz-Romero K, Perez A, Illanes S E, Mitchell M D, Rice G E, et al. Placenta-derived exosomes continuously increase in maternal circulation over the first trimester of pregnancy. Journal of Translational Medicine. 2014; 12(1):204.
3. Germain S J, Sacks G P, Soorana S R, Sargent I L, Redman C W. Systemic Inflammatory Priming in Normal Pregnancy and Preeclampsia: The Role of Circulating Syncytiotrophoblast Microparticles. The Journal of Immunology. 2007; 178(9):5949.
4. Tersigni C, Furqan Bari M, Cai S, Zhang W, Kandzija N, Buchan A, et al. Syncytiotrophoblast-derived extracellular vesicles carry apolipoprotein-E and affect lipid synthesis of liver cells in vitro. J Cell Mol Med. 2022; 26(1):123-32.
5. Cronqvist T, Erlandsson L, Tannetta D, Hansson S R. Placental syncytiotrophoblast extracellular vesicles enter primary endothelial cells through clathrin-mediated endocytosis. Placenta. 2020; 100:133-41.
6. Göhner C, Plösch T, Faas MM. Immune-modulatory effects of syncytiotrophoblast extracellular vesicles in pregnancy and preeclampsia. Placenta. 2017; 60 Suppl 1:S41-s51.
7. Hypertension in pregnancy: diagnosis and management. National Institute for Health and Care Excellence (NICE); 2019.
8. Salomon C, Guanzon D, Scholz-Romero K, Longo S, Correa P, Illanes S E, et al. Placental Exosomes as Early Biomarker of Preeclampsia: Potential Role of Exosomal MicroRNAs Across Gestation. J Clin Endocrinol Metab. 2017; 102(9):3182-94.
9. Pillay P, Maharaj N, Moodley J, Mackraj I. Placental exosomes and pre-eclampsia: Maternal circulating levels in normal pregnancies and, early and late onset pre-eclamptic pregnancies. Placenta. 2016; 46:18-25.
10. Goswami D, Tannetta D S, Magee L A, Fuchisawa A, Redman C W, Sargent I L, et al. Excess syncytiotrophoblast microparticle shedding is a feature of early-onset pre-eclampsia, but not normotensive intrauterine growth restriction. Placenta. 2006; 27(1):56-61.
11. Sammar M, Dragovic R, Meiri H, Vatish M, Sharabi-Nov A, Sargent I, et al. Reduced placental protein 13 (PP13) in placental derived syncytiotrophoblast extracellular vesicles in preeclampsia—A novel tool to study the impaired cargo transmission of the placenta to the maternal organs. Placenta. 2018; 66:17-25.
12. Awoyemi T, Tannetta D, Zhang W, Kandzija N, Motta-Mejia C, Fischer R, et al. Glycosylated Siglec-6 expression in syncytiotrophoblast-derived extracellular vesicles from preeclampsia placentas. Biochem Biophys Res Commun. 2020; 533(4):838-44.
13. Gill M, Motta-Mejia C, Kandzija N, Cooke W, Zhang W, Cerdeira A S, et al. Placental Syncytiotrophoblast-Derived Extracellular Vesicles Carry Active NEP (Neprilysin) and Are Increased in Preeclampsia. Hypertension. 2019; 73(5):1112-9.
14. Motta-Mejia C, Kandzija N, Zhang W, Mhlomi V, Cerdeira A S, Burdujan A, et al. Placental Vesicles Carry Active Endothelial Nitric Oxide Synthase and Their Activity is Reduced in Preeclampsia. Hypertension. 2017; 70(2):372-81.
15. Kabe Y, Suematsu M, Sakamoto S, Hirai M, Koike I, Hishiki T, et al. Development of a Highly Sensitive Device for Counting the Number of Disease-Specific Exosomes in Human Sera. Clin Chem. 2018; 64(10):1463-73.
16. Dragovic R A, Collett G P, Hole P, Ferguson D J, Redman C W, Sargent I L, et al. Isolation of syncytiotrophoblast microvesicles and exosomes and their characterisation by multicolour flow cytometry and fluorescence Nanoparticle Tracking Analysis. Methods. 2015; 87:64-74.
17. Kabe Y, Sakamoto S, Hatakeyama M, Yamaguchi Y, Suematsu M, Itonaga M, et al. Application of high-performance magnetic nanobeads to biological sensing devices. Anal Bioanal Chem. 2019; 411(9):1825-37.
18. Chen Y, Huang Y, Jiang R, Teng Y. Syncytiotrophoblast-derived microparticle shedding in early-onset and late-onset severe pre-eclampsia. Int J Gynaecol Obstet. 2012; 119(3):234-8.
19. Salomon C, Torres M J, Kobayashi M, Scholz-Romero K, Sobrevia L, Dobierzewska A, et al. A gestational profile of placental exosomes in maternal plasma and their effects on endothelial cell migration. PLoS One. 2014; 9(6):e98667.
20. Baranyai T, Herczeg K, Onódi Z, Voszka I, Módos K, Marton N, et al. Isolation of Exosomes from Blood Plasma: Qualitative and Quantitative Comparison of Ultracentrifugation and Size Exclusion Chromatography Methods. PLoS One. 2015; 10(12):e0145686.
21. Yamamoto K, Rakugi H. Angiotensin receptor-neprilysin inhibitors: Comprehensive review and implications in hypertension treatment. Hypertens Res. 2021; 44(10):1239-50.
22. Goodman O B, Jr., Febbraio M, Simantov R, Zheng R, Shen R, Silverstein R L, et al. Neprilysin inhibits angiogenesis via proteolysis of fibroblast growth factor-2. J Biol Chem. 2006; 281(44):33597-605.
23. Carrasco-Wong I, Aguilera-Olguin M, Escalona-Rivano R, Chiarello D I, Barragan-Zúñiga LJ, Sosa-Macias M, et al. Syncytiotrophoblast stress in early onset preeclampsia: The issues perpetuating the syndrome. Placenta. 2021; 113:57-66.
24. Fortunato D, Mladenovid D, Criscuoli M, Loria F, Veiman K L, Zocco D, et al. Opportunities and Pitfalls of Fluorescent Labeling Methodologies for Extracellular Vesicle Profiling on High-Resolution Single-Particle Platforms. Int J Mol Sci. 2021; 22(19).
25. Park F, Russo K, Williams P, Pelosi M, Puddephatt R, Walter M, et al. Prediction and prevention of early-onset pre-eclampsia: impact of aspirin after first-trimester screening. Ultrasound Obstet Gynecol. 2015; 46(4):419-23.
26. Roberge S, Nicolaides K H, Demers S, Villa P, Bujold E. Prevention of perinatal death and adverse perinatal outcome using low-dose aspirin: a meta-analysis. Ultrasound Obstet Gynecol. 2013; 41(5):491-9.
27. Roberge S, Villa P, Nicolaides K, Giguére Y, Vainio M, Bakthi A, et al. Early administration of low-dose aspirin for the prevention of preterm and term preeclampsia: a systematic review and meta-analysis. Fetal Diagn Ther. 2012; 31(3):141-6.

1. A method for prognosing an increased risk of early onset preeclampsia (EOPE) in a pregnant subject before the disease onset, the method comprising quantifying the detected absolute numbers of small placental extracellular vesicles (sEVs) that express three pairs of biomarkers, wherein the pairs of biomarkers are CD10+CD63+, CD10+placental alkaline phosphatase (PLAP)+, and CD63+PLAP+, in a plasma or serum sample obtained from the subject, and comparing the quantity of CD10+CD63+, CD10+PLAP+ and CD63+PLAP+ sEVs in the sample with reference quantities of CD10+CD63+, CD10+PLAP+, and CD63+PLAP+ sEVs, wherein an increased quantity of CD10+CD63+, CD10+PLAP+ or CD63+PLAP+ sEVs in the sample compared to the reference quantities indicates that the subject has an increased risk of developing EOPE.

2. The method of claim 1, wherein the method comprises
(I) capturing sEVs from the sample using sEV capture antibodies against (i) CD10 or CD63, (ii) CD10 or PLAP, and (iii) CD63 or PLAP; and incubating sEVs from the sample with sEV detection antibodies against the other biomarker of each pair (i) CD10 or CD63, (ii) CD10 or PLAP, and (iii) CD63 or PLAP, respectively, wherein each detection antibody is bound to a detectable moiety; and
(II) detecting the detectable moieties bound to captured sEVs to quantify the sEVs in the sample that express each pair of biomarkers.

3. The method of claim 2, wherein the method comprises:
(I) incubating the plasma or serum sample obtained from the subject with the anti-CD63, anti-CD10 and/or anti-PLAP sEV capture antibodies, wherein the capture antibodies are immobilised on a solid support, and wherein the incubation conditions are suitable for binding of sEVs in the sample to the capture antibodies;
(II) washing unbound sample from the solid support;
(III) incubating the captured sEVs with the detection antibodies bound to a detectable moiety under conditions suitable for binding of the detection antibodies to captured sEVs;
(IV) washing un-bound detection antibody from the solid support; and
(V) detecting the detectable moieties bound to the solid support via a detection antibody, sEV and capture antibody, to quantify the sEVs in the sample that express each pair of biomarkers.

4. The method of claim 1, wherein the method comprises
(I) incubating the plasma or serum sample obtained from the subject with anti-CD63, anti-CD10 and/or anti-PLAP sEV-capture antibodies, wherein the capture antibodies are coated on the surface of an optical disc comprising a plurality of wells, wherein the base of each well comprises a plurality of grooves, wherein the capture antibodies are coated on the base of the grooves, wherein each of the wells is coated with one of the sEV capture antibodies, anti-CD63, anti-CD10 or anti-PLAP, and wherein the incubation conditions are suitable for binding of sEVs in the sample to the capture antibodies in the grooves;
(II) washing un-bound sample from the disk;
(III) incubating the captured sEVs in the grooves with detection nanobeads, wherein the detection nanobeads are coated with anti-CD63, anti-CD10 or anti-PLAP sEV-detection antibodies, wherein the target antigen (CD63, CD10 or PLAP) of the capture antibodies in a well is different from the target antigen (CD63, CD10 or PLAP) of the sEV-detection antibodies coated on the nanobeads incubated in the same well, and wherein the incubation conditions are suitable for binding of detection antibody coated on single nanobeads to single sEVs in the grooves;
(IV) washing un-bound nanobeads from the disk; and
(V) counting the number of nanobeads bound to sEV in the grooves to quantify the sEVs in the sample that express each pair of biomarkers.

5. (canceled)

6. The method of claim 1, wherein the method uses pairs of sEV-capture:sEV-detection antibodies comprising anti-CD10:anti-CD63, anti-CD10:anti-PLAP and/or anti-CD63:anti-PLAP antibodies.

7. The method of claim 4, wherein the base of the grooves are about 160 nm in width.

8. The method of claim 4, wherein the detection nanobeads are about 200 nm in diameter.

9. The method of claim 4, wherein the top of the grooves are about 260 nm in width.

10. The method of claim 4, wherein the nanobeads are light-reflective nanobeads and wherein the method comprises counting the number of nanobeads in a groove by illuminating the groove using a laser diode and detecting light reflected from the beads.

11. The method of claim 10, wherein the nanobeads are magnetic light-reflective nanobeads, optionally wherein the nanobeads comprise a ferrite core, and further optionally comprise a resin coat, further optionally wherein the resin is glycidyl methacrylate (polyGMA).

12. The method of claim 4, wherein the method uses about 0.1% casein in phosphate-buffered saline containing 0.05% Tween-20 (PBST) as a nanobead-detection antibody dilution buffer for the incubation with the captured sEVs.

13. The method of claim 4, wherein the method comprises incubating the disc grooves comprising the capture antibodies with blocking buffer before incubation with the plasma or serum sample, and wherein the blocking buffer is about 0.1% casein in phosphate-buffered saline containing 0.05% Tween-20 (PBST).

14. The method of claim 4, wherein the method comprises the step of coating the surface of the optical disc with the capture antibodies, optionally wherein the capture antibodies are bound to the optical disc surface by passive absorption.

15. The method of claim 14, wherein the method further comprises washing unbound capture antibodies from the optical disc.

16. The method according to claim 1, and further comprising administering prophylactic treatment for EOPE to the subject determined to be at increased risk of EOPE.

17. A method for preventing early onset preeclampsia (EOPE) in a pregnant subject, wherein the method comprises administering prophylactic treatment for EOPE to the subject, and wherein the subject has been prognosed as being at increased risk of EOPE using a method according to claim 1.

18. The method of claim 17, wherein the prophylactic treatment is administration of aspirin.

19-21. (canceled)

22. A product comprising
(a) an optical disc, wherein the optical disc comprises a plurality of wells for holding a serum or plasma sample, wherein the base of each well comprises a plurality of grooves, wherein the base of the grooves are about 160 nm in width and the top of the grooves are about 260 nm in width, wherein the base of the grooves in a well of the disc is coated with anti-CD10 antibodies and wherein the base of the grooves in a different well of the disc is coated with anti-CD63 antibodies; or
(b) a kit comprising the optical disc of (b) and two sets of light-reflective nanobeads, wherein the nanobeads of one of the two sets are coated with anti-placental alkaline phosphatase (PLAP) antibodies and the nanobeads of the other set are coated with anti-CD63 antibodies.

23. (canceled)
